(19) United States
(12) Patent Application Publication
(10) Pub. No.: US 2022/0135940 A1
(43) Pub. Date: May 5, 2022

(54) METHOD FOR PRODUCING KIDNEY STRUCTURE HAVING DENDRITICALLY BRANCHED COLLECTING DUCT FROM PLURIPOTENT STEM CELLS

(71) Applicant: NATIONAL UNIVERSITY CORPORATION KUMAMOTO UNIVERSITY, Kumamoto (JP)

(72) Inventors: Ryuichi NISHINAKAMURA, Kumamoto (JP); Atsuhiro TAGUCHI, Kumamoto (JP)

(21) Appl. No.: 17/292,595

(22) PCT Filed: Nov. 8, 2018
(86) PCT No.: PCT/JP2018/041558

§ 371 Date: Dec. 23, 2021

(51) Int. Cl. C12N 5/0735

(52) U.S. Cl. CPC C12N 5/0606; C12N 2501/105; C12N 2501/415; C12N 2501/12; C12N 2501/30; C12N 2501/117

(57) ABSTRACT

An exemplary method can be provided for producing ureteric bud cells from pluripotent stem cells, in vitro. Further, an exemplary method can be provided for producing ureteric bud cells and Wolffian duct (WD) progenitor-like cells that are a progenitor of the ureteric bud cells. Another exemplary method can be provided for producing Wolffian duct (WD) progenitor-like cells that can comprise obtaining C-X-C chemokine receptor 4 (CXCR4) positive and KIT proto-oncogene receptor tyrosine kinase (KIT) positive cells. In addition, an exemplary method can be provided for producing ureteric-bud-like cells using WD progenitors that are CXCR4+ and KIT+, and another exemplary method can be provided for producing kidney organoids in which the ureteric-bud-like cells can be used.

### CROSS REFERENCE TO RELATED APPLICATION(S)

This application relates to, and claims the benefit and priority from International Patent Application No. PCT/JP2018/041558 filed on Nov. 8, 2018, and published as International Publication WO 2020/095423 on May 14, 2020, the entire disclosures of all of which are incorporated herein by reference in their entireties.

### FIELD OF THE DISCLOSURE

The present disclosure relates a method for producing higher-order kidney structure from pluripotent stem cells. More particularly, a method for inducing branched collecting duct structure via Wolffian duct (WD) progenitor-like cells differentiation followed by ureteric bud (UB)-like tissue formation procedures. The method for producing WD progenitor-like cells comprises a procedure of purifying C-X-C chemokine receptor 4 (CXCR4)-positive and KIT proto-oncogene receptor tyrosine kinase (KIT)-positive cells.

### BACKGROUND INFORMATION

The kidney develops from three types of progenitor cells and blood vessels. The progenitors comprise (1) nephron progenitors, which differentiate into nephrons; the blood filtration/urine production units, (2) stromal progenitors, which assist nephron formation and (3) ureteric bud, which differentiates into collecting ducts; the urine drainage units. Among them, (1) and (2) are collectively referred as metanephric mesenchyme (MM) based on their close proximity in origin.

The kidney produces urine by filtrating blood in millions of nephrons. Produced urine will be drained by collecting ducts, which connect all the nephrons to the single exit, that is, the ureter. To construct this dendritically branched collecting duct structure, ureteric bud must possess capacity to undergo successive bifurcation at the tips.

The tips of the ureteric bud also serves for the progenitor niche formation during embryonic organogenesis, which is essential to develop the organ to functionally sufficient size. However, the method of inducing ureteric bud, which equips these functional features, from pluripotent stem cells has not been reported.

It was previously reported the successful induction of kidney nephron structures via directed differentiation of nephron progenitors from pluripotent stem cells.

Other reports have shown induction of collecting duct-like cells/tissues from pluripotent stem cells (Non-Patent Documents 1 and 2). However, none of these methods have reproduced the dendritically branched structure of the collecting duct, which is required to connect each nephron to the single exit. For example, the collecting duct-like cells presented in the Non-Patent Document 2 showed single tube like structure but lacked consecutive branching.

### OBJECTIVES AND SUMMARY OF EXEMPLARY EMBODIMENTS

The present disclosure has an exemplary object of providing methods for producing WD progenitor-like cells capable of being differentiated into ureteric bud-like cells, and producing collecting duct-like structures.

For example, the signals required for the process of ureteric bud differentiation have been identified. Identification of cell surface antigens enabled sorting and purification of WD progenitor-like cells that can further differentiate into ureteric bud-like cells. With these exemplary embodiments of the present disclosure, it is possible to induce ureteric but-like cells from mouse ES cells and human iPS cells. When co-cultured the induced ureteric bud with nephron progenitors and stromal progenitors, it reproduced the higher-order structure of the fetal kidney, including dendritically branching collecting duct structures and progenitor niche formation.

Further investigations have been performed intensively based on this finding, leading to completion of the present disclosure.

To that end, the exemplary embodiments of the present disclosure comprise the following:

[1] A method for producing Wolffian duct (WD) progenitor-like cells, comprising (Procedure A) a procedure of obtaining C-X-C motif chemokine receptor 4 (CXCR4)-positive and KIT proto-oncogene receptor tyrosine kinase (KIT)-positive cells.

[2] The method according to [1], wherein procedure of obtaining the CXCR4-positive and KIT-positive cells is a procedure of sorting cells in which the proportion of the CXCR4-positive and KIT-positive cells with respect to all cells is 30% or more, preferably 50% or more, more preferably 70% or more, further preferably 80% or more and more further preferably 90% or more.

[3] The method according to [1], wherein the CXCR4-positive and KIT-positive cells further express at least 2, preferably at least 3 and further preferably all selected from the group consisting of Paired box (PAX) 2, LIM homeobox (LHX) 1, empty spiracles homeobox (EMX) 2, ret proto-oncogene (RET) and homeobox (HOX) B7.

[4] The method according to any one of [1] to [3], further comprising the following procedures B1, B2, C and D:

(Procedure B1) a procedure of culturing pluripotent stem cells in a medium containing activin A or a tumor growth factor (Tgfb1 or Tgfb2) (preferably activin A),

(Procedure B2) a procedure of culturing cells obtained by procedure B1 in a medium containing a Wnt agonist (preferably Glycogen Synthase Kinase (GSK)-3β inhibitor, more preferably CHIR99021 or SB216763),

(Procedure C) a procedure of culturing cells obtained by procedure B2 in a medium containing retinoic acid (RA) or an RA analog (preferably RA or AGN193109), a fibroblast cell growth factor (FGF2, FGF4, FGF7, FGF9 or FGF20, preferably FGF9), and a TGFβ signal pathway inhibitor or a Wnt agonist (preferably SB431542 or A83-01), and

(Procedure D) a procedure of culturing cells obtained by procedure C in a medium containing RA or an RA analog (preferably RA or AGN193109), a Wnt agonist (preferably GSK-3β inhibitor, more preferably CHIR99021 or SB216763), and a fibroblast cell growth factor (FGF2, FGF4, FGF7, FGF9 or FGF20, preferably FGF9 or FGF20, more preferably FGF9)

(Wherein, the components in each procedure may be the same substance or difference substances).

[5] The method according to [4], wherein procedure A is performed by sorting CXCR4-positive and KIT-positive cells from cells obtained by procedure D.

[6] The method according to [4] or [5], wherein the pluripotent stem cells are embryonic stem cells.

[7] The method according to [4] or [5], wherein the pluripotent stem cells are iPS cells.

[8] The method according to [4] or [5], wherein the pluripotent stem cells are human iPS cells.

[9] The method according to any one of [4] to [8], wherein the medium used in procedure B1 contains 1 ng/mL to 1000 ng/mL, preferably 1 ng/mL to 100 ng/mL, and more preferably 3 to 30 ng/mL of activin A.

[10] The method according to any one of [4] to [9], wherein the medium used in procedure B1 further contains a BMP signal pathway active substance (preferably BMP2, BMP4 or BMP7, more preferably BMP2 or BMP4, and further preferably BMP4).

[11] The method according to any one of [4] to [9], wherein the medium used in procedure B1 contains 10 ng/mL or less, preferably 0.1 ng/mL to 10 ng/mL, more preferably 0.3 ng/mL to 3 ng/mL, and further preferably about 1 ng/mL of BMP4.

[12] The method according to any one of [4] to [11], wherein the Wnt agonist in procedures B2 and D is CHIR99021 or SB216763 (preferably CHIR99021), respectively.

[13] The method according to any one of [4] to [11], wherein the medium used in procedure B2 contains 1 μM to 1000 μM, preferably 1 μM to 200 μM, more preferably 3 μM to 30 μM, and further preferably about 10 μM of CHIR99021.

[14] The method according to any one of [4] to [13], wherein the medium used in procedure B2 further contains a BMP signal pathway active substance (preferably BMP2, BMP4 or BMP7, more preferably BMP2 or BMP4, further preferably BMP4).

[15] The method according to [10] or [14], wherein the BMP signal pathway active substance is BMP4.

[16] The method according to any one of [4] to [13], wherein the medium used in procedure B2 contains 10 ng/mL or less, preferably 5 ng/mL or less, and more preferably 0.3 ng/mL to 3 ng/mL of BMP4.

[17] The method according to any one of [4] to [16], wherein the culturing time in procedure B2 is about 1 day to 2 days.

[18] The method according to any one of [4] to [16], wherein the culturing time in procedure B2 is about 1.5 days.

[19] The method according to any one of [4] to [16], wherein the culturing time in procedure B1 is about 1 day.

[20] The method according to any one of [4] to [16], wherein the culturing time in procedure B1 is about 1 day and the culturing time in procedure B2 is about 1.5 days.

[21] The method according to any one of [4] to [20], wherein the TGFβ signal pathway inhibitor or the Wnt agonist in procedure C is a TGFβ signal pathway inhibitor (preferably SB431542 or A83-01).

[22] The method according to any one of [4] to [20], wherein the medium used in procedure C contains 1 μM to 1000 μM, preferably 3 μM to 500 μM, and more preferably 10 μM to 200 μM of SB431542.

[23] The method according to any one of [4] to [22], wherein the medium used in procedure C contains 10 nM to 1 μM, preferably 10 to 500 nM, more preferably 50 nM to 200 nM, and further preferably about 100 nM of retinoic acid.

[24] The method according to any one of [4] to [23], wherein the medium used in procedure C contains 10 ng to 1 μg/mL, preferably 10 ng to 500 ng/mL, more preferably 50 ng to 200 ng/mL, and further preferably about 100 ng/mL of FGF9.

[25] The method according to any one of [4] to [24], wherein the WD progenitor-like cells re human cells, and the medium used in procedure C further contains a BMP signal pathway inhibitor.

[26] The method according to [25], wherein the BMP signal pathway inhibitor used in procedure C is LDN193189 or Noggin.

[27] The method according to [26], wherein the medium used in procedure C contains 1 nM to 1000 nM, preferably 3 nM to 500 nM, more preferably 10 nM to 200 nM, and further preferably about 100 nM of LDN193189.

[28] The method according to any one of [4] to [27], wherein the culturing time in procedure C is about 1 to 3 days, and preferably about 1 to 2 days.

[29] The method according to any one of [4] to [28], wherein the medium used in procedure D contains 10 nM to 1 μM, preferably 10 to 500 nM, more preferably 50 nM to 200 nM, and further preferably about 100 nM of retinoic acid.

[30] The method according to any one of [4] to [29], wherein the medium used in procedure D contains 0.1 μM to 100 μM, preferably 1 μM to 100 μM, more preferably 1 μM to 10 μM, and further preferably about 3 μM to 5 μM of CHIR99021.

[31] The method according to any one of [4] to [30], wherein the medium used in procedure D contains 10 ng to 1 μg/mL, preferably 10 ng to 500 ng/mL, more preferably 30 ng to 300 ng/mL, and further preferably about 100 ng/mL of FGF9.

[32] The method according to any one of [4] to [31], wherein the WD progenitor-like cells are human cells, and the medium used in procedure D further contains a BMP signal pathway inhibitor.

[33] The method according to [32], wherein the BMP signal pathway inhibitor used in procedure D is LDN193189 or Noggin.

[34] The method according to [33], wherein the medium used in procedure D contains 1 nM to 500 nM, preferably 10 nM to 100 nM, more preferably 10 nM to 50 nM, and further preferably about 30 nM of LDN193189.

[35] The method according to any one of [4] to [34], wherein the culturing time in procedure D is about 1 to 3 days, and preferably about 1.5 days to about 2.5 days.

[36] A method for producing ureteric bud-like cells, comprising (Procedure E) a procedure of culturing CXCR4-positive and KIT-positive WD progenitor-like cells in a medium containing RA or an RA analog (preferably RA or AGN193109), a Wnt agonist (preferably GSK-33 inhibitor or Rspondin1, more preferably CHIR99021, SB216763 or Rspondin1), a fibroblast cell growth factor (FGF2, FGF4, FGF7, FGF9 or FGF20, preferably FGF9) and an ROCK inhibitor (preferably Y27632 or Fasudil hydrochloride).

[37] The method according to [36], wherein the CXCR4-positive and KIT-positive WD progenitor-like cells are cells obtained in the method as described in [1] or [2].

[38] The method according to [36] or [37], wherein the medium used in procedure (E) contains 0.1 ng/mL to 100 ng/mL, preferably 0.5 ng/mL to 50 ng/mL, more preferably 2 ng/mL to 10 ng/mL, and further preferably about 5 ng/mL of FGF9.

[39] The method according to any one of [36] to [38], wherein the medium used in procedure (E) contains 10 nM to 1 μM, preferably 10 nM to 500 nM, more preferably 50 nM to 200 nM, and further preferably about 100 nM of RA.

[40] The method according to any one of [36] to [39], wherein the ROCK inhibitor is Y27632.

[41] The method according to any one of [36] to [39], wherein the medium used in procedure (E) contains 1 μM to 1000 μM, preferably 1 μM to 100 μM, more preferably 1 μM to 50 μM, and further preferably about 10 μM of Y27632.

[42] The method according to any one of [36] to [41], wherein the Wnt agonist is CHIR99021 or Rspondin1.

[43] The method according to any one of [36] to [41], wherein the medium used in procedure (E) contains 0.1 μM to 100 μM, preferably 0.1 μM to 10 μM, more preferably 0.3 μM to 5 μM, and further preferably about 1 μM of CHIR99021.

[44] The method according to any one of [36] to [43], wherein the WD progenitor-like cells are human cells, and the medium used in procedure (E) further contains FGF1 and a BMP signal pathway inhibitor (preferably LDN193189 or Noggin).

[45] The method according to [44], wherein the medium used in procedure (E) contains 10 ng/mL to 1000 ng/mL, preferably 10 ng/mL to 500 ng/mL, more preferably 50 ng/mL to 200 ng/mL, and further preferably about 100 ng/mL of FGF1.

[46] The method according to any one of [44] to [45], wherein the BMP signal pathway inhibitor is LDN193189.

[47] The method according to any one of [44] to [45], wherein the medium used in procedure (E) contains 1 nM to 300 nM, preferably 1 nM to 100 nM, more preferably 1 nM to 20 nM, and further preferably about 10 nM of LDN193189.

[48] The method according to any one of [36] to [47], further comprising (Procedure F) a procedure of culturing cells obtained by procedure (E) in a medium containing RA or an RA analog (preferably RA or AGN193109), a Wnt agonist (preferably GSK-33 inhibitor or Rspondin1, more preferably CHIR99021, SB216763 or Rspondin1), a fibroblast cell growth factor (FGF2, FGF4, FGF7, FGF9 or FGF20, preferably FGF9), an ROCK inhibitor (preferably Y27632 or Fasudil hydrochloride), and a glial cell line-derived neurotrophic factor (GDNF) or a GDNF analog (preferably BT18) or FGF10.

[49] The method according to any one of [36] to [47], wherein the medium used in procedure (F) contains 0.1 ng to 100 ng/mL, preferably 0.1 ng to 10 ng/mL, more preferably 0.5 ng to 10 ng/mL, and further preferably about 1 ng/mL of GDNF.

[50] The method according to [48] or [49], wherein the medium used in procedure (F) contains 0.1 ng/mL to 100 ng/mL, preferably 0.5 ng/mL to 50 ng/mL, more preferably 2 ng/mL to 10 ng/mL, and further preferably about 5 ng/mL of FGF9.

[51] The method according to any one of [48] to [50], wherein the medium used in procedure (F) contains preferably 10 nM to 1 μM, preferably 10 nM to 500 nM, more preferably 50 nM to 200 nM, and further preferably about 100 nM of RA.

[52] The method according to any one of [48] to [51], wherein the ROCK inhibitor in procedure (F) is Y27632.

[53] The method according to any one of [48] to [51], wherein the medium used in procedure (F) contains 1 μM to 1000 μM, preferably 1 μM to 100 μM, more preferably 1 μM to 50 μM, and further preferably about 10 μM of Y27632.

[54] The method according to any one of [48] to [53], wherein the Wnt agonist in procedure (F) is CHIR99021.

[55] The method according to any one of [48] to [53], wherein the medium used in procedure (F) contains 0.1 μM to 300 μM, preferably 0.3 μM to 100 μM, more preferably 1 μM to 5 μM, and further preferably about 3 μM of CHIR99021.

[56] The method according to any one of [48] to [55], wherein the WD progenitor-like cells are human cells, and the medium used in procedure (F) further contains FGF1 and a BMP signal pathway inhibitor (preferably LDN193189 or Noggin).

[57] The method according to [56], wherein the medium used in procedure (F) contains 10 ng/mL to 1000 ng/mL, preferably 10 ng/mL to 500 ng/mL, more preferably 50 ng/mL to 200 ng/mL, and further preferably about 100 ng/mL of FGF1.

[58] The method according to [56] or [57], wherein the BMP signal pathway inhibitor is LDN193189.

[59] The method according to [56] or [57], wherein the medium used in procedure (F) contains 1 nM to 300 nM, preferably 1 nM to 100 nM, more preferably 5 nM to 20 nM, and further preferably about 10 nM of LDN193189.

[60] The method according to any one of [48] to [59], further comprising (Procedure G) a procedure of culturing cells obtained by procedure (F) in a medium containing RA or an RA analog (preferably RA or AGN193109), a Wnt agonist (preferably GSK-33 inhibitor or Rspondin1, more preferably CHIR99021, SB216763 or Rspondin1), an ROCK inhibitor (preferably Y27632 or Fasudil hydrochloride), and GDNF or a GDNF analog (preferably BT18).

[61] The method according to [60], wherein the medium used in procedure (G) contains 0.1 ng to 100 ng/mL, preferably 0.2 ng to 20 ng/mL, more preferably 0.5 ng to 10 ng/mL, and further preferably about 2 ng/mL of GDNF.

[62] The method according to [60] or [61], wherein the medium used in procedure (G) contains 10 nM to 1 μM, preferably 10 nM to 500 nM, more preferably 50 nM to 200 nM, and further preferably about 100 nM of RA.

[63] The method according to any one of [60] to [62], wherein the ROCK inhibitor in procedure (G) is Y27632.

[64] The method according to any one of [60] to [62], wherein the medium used in procedure (G) contains 1 μM to 1000 μM, preferably 1 μM to 100 μM, more preferably 1 μM to 50 μM, and further preferably about 10 μM of Y27632.

[65] The method according to any one of [60] to [64], wherein the Wnt agonist in procedure (G) is CHIR99021.

[66] The method according to any one of [60] to [64], wherein the medium used in procedure (G) contains 0.1 μM to 300 μM, preferably 0.3 μM to 100 μM, more preferably 1 μM to 5 μM, and further preferably about 3 μM of CHIR99021.

[67] The method according to any one of [60] to [66], wherein the WD progenitor-like cells are human cells, and the medium used in procedure (G) further contains FGF1 and a BMP signal pathway inhibitor (preferably LDN193189 or Noggin).

[68] The method according to [67], wherein the medium used in procedure (G) contains 10 ng/mL to 1000 ng/mL, preferably 10 ng/mL to 500 ng/mL, more preferably 50 ng/mL to 200 ng/mL, and further preferably about 100 ng/mL of FGF1.

[69] The method according to [67] or [68], wherein the BMP signal pathway inhibitor is LDN193189.

[70] The method according to [67] or [68], wherein the medium used in procedure (G) contains 1 nM to 300 nM, preferably 1 nM to 100 nM, more preferably 5 nM to 20 nM, and further preferably about 10 nM of LDN193189.

[71] A method for producing a kidney organoid, comprising co-culturing ureteric bud-like cells induced from CXCR4-positive and KIT-positive WD progenitor-like cells with nephron progenitor cells, and embryonic kidney-derived Platelet Derived Growth Factor Receptor Alpha (PDGFRa)-positive stromal cell population.

[72] The method according to [71], wherein the WD progenitor-like cells are WD progenitor-like cells produced by the method as described in any one of [2] to [35].

[73] The method according to any one of [36] to [70], wherein the ureteric bud-like cells express HNF1b, E-Cadherin and CALB1.

[74] The method according to any one of [36] to [70], wherein the ureteric bud-like cells express EMX2, WNT11, HNF1b, E-Cadherin and CALB1.

[75] A kit for producing WD progenitor-like cells from pluripotent stem cells, comprising

a medium B1 containing activin A,

a medium B2 containing a Wnt agonist (preferably GSK-33 inhibitor, more preferably CHIR99021 or SB216763),

a medium C containing RA or an RA analog, a fibroblast cell growth factor (any of FGF2, FGF9 or FGF20) and a TGFβ signal pathway inhibitor (preferably SB431542 or A83-01), and

a medium D containing RA or an RA analog, a Wnt agonist (preferably GSK-3β inhibitor, more preferably CHIR99021 or SB216763) and a fibroblast cell growth factor (any of FGF2, FGF9 or FGF20).

[76] A kit for producing ureteric bud-like cells from pluripotent stem cells, comprising the kit as described in [75], and a medium E containing RA or an RA analog, a Wnt agonist (preferably GSK-3β inhibitor or Rspondin1, more preferably CHIR99021, SB216763 or Rspondin1), a fibroblast cell growth factor (any of FGF2, FGF9 or FGF20), and an ROCK inhibitor (preferably Y27632 or Fasudil hydrochloride).

[77] The kit according to [75] or [76], further comprising an anti-CXCR4 antibody and an anti-KIT antibody.

According to the method provided by the present disclosure, it can become possible to produce ureteric bud-like cells in vitro, which has equivalent functional capacity to the embryonic ureteric bud. More particularly, induced ureteric bud-like cell is capable of forming dendritic branching structures, maintaining a progenitor cell niche at the tip of the branch, and connecting to individual nephrons within a kidney organoid. According to the present disclosure, sorting CCXR4-positive and KIT-positive cells enables to obtain progenitor cells capable of differentiating into functional ureteric bud-like cells, which can constitute a branched collecting duct structures. Further, according to the method according to the exemplary embodiment(s) of the present disclosure, it becomes possible to obtain CXCR4-positive and KIT-positive cells with high efficiency from pluripotent stem cells, and that makes it possible to induce functional ureteric bud-like cells.

According to the method for producing WD progenitor-like cells provided in the present disclosure, it can become possible to produce ureteric bud-like cells having following features, which were not observed in the previously reported pluripotent stem cells-derived ureteric bud-like cells. (I) When mixed with metanephric mesenchyme or cultured in a growth factor which promotes branching, they undergoes branching. (II) When mixed with metanephric mesenchyme, they show an ability of forming a kidney progenitor niche, which maintains undifferentiated nephron progenitors. (III) When mixed with metanephric mesenchyme, they show an ability of differentiating nephron progenitor cells into the nephrons. In the presence of nephron progenitors and stromal progenitors, the ureteric bud-like cells produced using the method provided by the present disclosure can form a kidney organoid, which contains differentiated nephrons connected by dendritically branching collecting duct structures. The kidney organoid described herein as exemplary embodiments shows the first evidence of reproducing the higher-order structure of the kidney in the world. Hence, the present disclosure can be an indispensable technique for enabling production of a functional artificial kidney in the future.

### BRIEF DESCRIPTION OF DRAWINGS

FIG. 1 is a view showing the outline of the protocol for the production of higher-order structure of embryonic kidney, which employs differentially induced ureteric bud and nephron progenitor cells from pluripotent stem cells. The upper panels show the present disclosure; the method of producing Wolffian duct (WD) progenitor-like cells and the following ureteric bud induction procedures. The lower panels show the outline of the protocol for inducing nephron progenitor cells.

FIG. 2 is a view showing an outline of the protocol for inducing ureteric bud from mouse embryonic stem cells. A10: 10 ng/mL activin A; B0.3: 0.3 ng/mL Bmp4; C5: 5 μM CHIR; C10: 10 μM CHIR; R: 0.1 μM retinoic acid; F9-100: 100 ng/mL Fgf9; SB10: 10 μM SB431542; Lif-: not containing leukemia inhibitory factor (LIF); Y: Y27632; C: CHIR; F: Fgf9; G: GDNF. In the figure, concentrations and periods are described as a preferred exemplary embodiment in the practice of the present disclosure, but the present disclosure is not limited thereto.

FIG. 3 is a view showing an outline of the protocol for inducing ureteric bud from human iPS cells. A10: 10 ng/mL activin A; B1: 1 ng/mL Bmp4; C1, C3, C5, C10: 1, 3, 5 or 10 μM CHIR, respectively; R: 0.1 μM retinoic acid; F9-100, F9-5: 100 or 5 ng/mL Fgf9, respectively; SB100: 100 μM SB431542; LDN10, 30, 100: 10, 30 or 100 nM LDN193189, respectively; Y: Y27632; G1, G2: each 1 ng/ml or 2 ng/ml GDNF. In the figure, concentrations and periods are described as a preferred exemplary embodiment in the practice of the present disclosure, but the present disclosure is not limited thereto.

FIG. 4 shows a schematic view of in vivo WD and ureteric bud differentiation process in mice. The parts of the WD used for the reconstruction assays or microarray analyses are outlined by the dashed lines. E: abbreviation of embryonic day.

FIG. 5 shows the quantified tip numbers of the reconstructed branching ureteric epithelium. *P<0.05 and **P<0.01.

FIG. 6 shows the gene expression kinetics by qRT-PCR analysis. The gene expression levels of the sorted WD or UB at each stage are presented. It shows relative gene expression levels of transcripts to β-actin expression (n=3).

FIG. 7 shows that retinoic acid, Wnt and Fgf/Gdnf signaling mature WD progenitor cells into ureteric bud. The relative expression levels of transcripts to β-actin expression is shown as the mean±s.e.m. (n=4). Y: Y27632 (Rock inhibitor); R: retinoic acid, C: 3 μM CHIR 99021 (canonical Wnt agonist), C1: 1 μM CHIR 99021, C3: 3 μM CHIR 99021, F: 100 ng/mL Fgf9, F5: 5 ng/mL Fgf9, G1: 1 ng/ml GDNF, G2: 2 ng/ml GDNF. The upper figure shows the result at day 2 of in vitro differentiation of the sorted E9.5 embryonic WD. The lower figure shows the result at day 1 of in vitro differentiation of the sorted E8.75 embryonic WD.

FIG. 8 shows a bright-field and GFP fluorescence images of an induced UB-like structure at day 3 of culture. Scale bars, 100 mm.

FIG. 9 shows the temporal kinetics of marker genes. The expression level in the in vivo WD is shown on the left side of each marker set. Relative expression levels of transcripts to b-actin expression are presented (n=4)

FIG. 10: Fluorescence-activated cell sorting (FACS) analyses of WD marker gene expression. Left panel: the Hoxb7−GFP+/Flk1− WD progenitor fraction is outlined. Right panel: analysis of CXCR4/KIT expression in Hoxb7−GFP+/Flk1− WD progenitors. The CXCR4+/KIT+ WD progenitor fraction is outlined.

Lower figure shows CXCR4 and KIT-strongly positive fraction (WD progenitor fraction) in the E8.75 embryo proper.

FIG. 11 shows the result of FACS analysis at day 6.25, by modulating the differentiation factor in procedure 4 (Procedure D of the method according to the exemplary embodiment(s) of the present disclosure). The result is shown as the means±s.e.m. (n=3). NO: no factor added; RC: 0.1 μM retinoic acid+5 μM CHIR99021; RF: 0.1 μM retinoic acid+100 ng/mL Fgf9; CF: 5 μM CHIR99021+100 ng/mL Fgf9; RCF: 0.1 μM retinoic acid+5 μM CHIR99021+100 ng/mL Fgf9.

FIG. 12 shows the result of FACS analysis at day 6.25 ureteric bud (UB) or day 8.5 metanephric mesenchyme (MM), by modulating the Bmp4 concentration in procedure 2 (Procedure B2 of the method according to the exemplary embodiment(s) of the present disclosure). The result is shown as the means±s.e.m. (n=3). BOC10: 0 ng/mL Bmp4+10 μM CHIR, B0.3C10: 0.3 ng/mL Bmp4+10 μM CHIR, B1C10: 1 ng/mL Bmp4+10 μM CHIR.

FIG. 13 shows the result of FACS analysis at day 6.25 (UB) or Day 8.5 (MM), by modulating the activin A concentration in procedure 1 (Procedure B1 of the method according to the exemplary embodiment(s) of the present disclosure). The result is shown as the means±s.e.m. (n=3). AO: 0 ng/mL activin A, A1: 1 ng/mL activin A, A3: 3 ng/mL activin A, A10: 10 ng/mL activin A, A30: 30 ng/mL activin.

FIG. 14 is a result showing optimization of the activin/Bmp concentration at the stage of epiblast patterning (procedure 1 and procedure 2), in the case of use of mouse ES cells. It shows an impact of the activin/Bmp concentration at the stage of epiblast patterning on the following fate determination of UB (upper table and graph) versus MM (lower table and graph).

FIG. 15 shows the temporal kinetics of marker genes from immature mouse ES cells at day 0 to WD progenitor stage at day 6.25 (corresponding to E8.75 WD progenitor). The expression levels in E8.75 embryonic WD progenitors are shown as triangles. Relative expression levels of transcripts to b-actin expression are presented (n=3).

FIG. 16 shows an outline of the protocol of differentiation of the sorted E8.75 WD progenitors into ureteric bud in vitro. Y: Y27632 (Rock inhibitor); R: 0.1 μM retinoic acid, C1: 1 μM CHIR 99021, C3: 3 μM CHIR 99021, F9-5: 5 ng/mL Fgf9, G1: 1 ng/ml GDNF, G2: 2 ng/ml GDNF.

FIG. 17 shows UB at day 9.25 of differentiation induced from mouse embryonic stem cells. Left panel: low magnification image of the whole spheroid. The fluorescent image is shown below. Right panel: enlarged view of induced UB isolated manually. The fluorescent image is shown on the right side. Scale bar: 100 μm.

FIG. 18 shows the temporal kinetics of marker genes of WD progenitors induced from mouse embryonic stem cells. The expression level in E11.5 embryonic UB is indicated by a triangle points. The relative expression levels of each transcript to β-actin expression are shown as the means±s.e.m. (n=3).

FIG. 19: Time-course images of the reconstructed organoid. Time points from day 1.5 (D1.5) to day 6 (D6) are shown. The fluorescent image is shown in lower panels. Arrows and numbers show the generation of the indicated bifurcations. Scale bar: 100 μm. The bar graph shows the total number of UB tips of organoids reconstructed from either induced UB or embryonic UB is shown as the means±s.e.m. (n=6). P=0.53.

FIG. 20 shows the result of immunostaining of organoids reconstructed from mouse embryonic kidney-derived metanephric mesenchyme and mouse ES cell-derived induced UB. The left view is a 3D projection image of an immuno-stained organoid at day 7. Upper panel: merged image of CK8 and Six2 staining. Lower panel: CK8 single-stained image. Scale bar, 100 μm. The right figure is a 3D projection image of an immuno-stained organoid at day 7. The respectively displayed molecules are stained by a single color (left four panels) or an merged image (upper right panel). Scale bar: 200 μm. The lower right figure shows an image of a section of an immuno-stained organoid at day 7. The UB tip and nephron junction region is enlarged. Scale bar: 20 μm.

FIG. 21 shows the result of immunostaining of organoids reconstructed from mouse ES cell-derived nephron progenitor cells, induced UB and embryonic stromal cells. Left two panels: organoid 3D projection image. Right two panels: organoid section.

FIG. 22 shows the effect of modulating differentiation factor in procedure 3 (Procedure C of the method according to the exemplary embodiment(s) of the present disclosure) of ureteric bud induction from human iPS cells. The left figure shows the expression of ureteric bud marker genes. The relative expression levels of each transcript to β-actin expression are analyzed at day 4.5 and shown as the means±s.e.m. (n=4). The right figure shows the result of FACS analysis at day 6.25. The induction rate of the CXCR4-positive/KIT-positive fraction is shown as the means±s.e.m. (n=3). R: 0.1 μM retinoic acid, F: 100 ng/mL Fgf9, L: LDN 100 nM, S: SB 100 μM.

FIG. 23 shows the result of UB or nephron progenitor induction efficiency from human iPS cells by modulating the activin A concentration in procedure 1 (Procedure B1 of the method according to the exemplary embodiment(s) of the present disclosure). The left figure shows the result of FACS analysis of the ureteric bud induction rate at day 6.25, and the right figure shows the result of FACS analysis of the nephron progenitor induction rate at day 12. In the Bmp4 plus condition, 1 ng/mL of Bmp4 was added. The result is shown as the means±s.e.m. (n=3).

FIG. 24 is a view showing optimization of the activin/Bmp concentration at the stage of epiblast patterning, in the case of human iPS cells differentiation. It shows the impact of activin/Bmp concentration at the stage of epiblast patterning (Procedure 1 and Procedure 2) (Procedure B1 and B2 of the method according to the exemplary embodiment(s) of the present disclosure) of human iPS cells on the following fate determination of UB (upper table and graph) versus MM (lower table and graph).

FIG. 25 shows the result of FACS analysis at day 6.25 (UB lineage) or day 12 (MM lineage), by modulating the Bmp concentration in procedure 2 (Procedure B2 of the method according to the exemplary embodiment(s) of the present disclosure). In the Bmp4 plus condition, 1 ng/mL of Bmp4 was added. The result is shown as the means±s.e.m. (n=3). L30C10: 30 nM LDN+10 μM CHIR, BOC10: 0 ng/mL Bmp4+10 μM CHIR, B1C10: 1 ng/mL Bmp4+10 μM CHIR.

FIG. 26 shows the result of FACS analysis of CXCR4/KIT expression at day 6.25 of human iPS cells induced under the optimized UB differentiation condition.

FIG. 27 shows the temporal kinetics of WD marker genes during the WD induction from human iPS cells under the optimized condition. The relative expression levels of each transcript to β-actin expression are shown as the means±s.e.m. (n=3).

FIG. 28 shows a bright field image of induced UB branching in the environment of 50% Matrigel culture. Scale bar, 200 μm.

FIG. 29 shows a 3D projection image of an immuno-stained organoid at day 13. The left figure shows an image stained with CK8 and SOX9. The right figure shows images stained with PAX2 and E-cadherin. Scale bar: 200 μm.

FIG. 30 is a view showing the cell-autonomous requirement of PAX2 in differentiation of human UB by utilizing wild type and PAX2 knock-out (KO) hiPSC. It shows bright field images of aggregates after maturation culture from day 8.5 to day 12.5 (left 6 panels), and aggregates at day 3 and day 12 of branching culture of sorted WD progenitors (right 4 panels).

FIG. 31 is a view showing the impact of different nephron differentiation methods on the finally-formed nephron numbers. Mouse metanephric mesenchymes were induced differentiation either with fetal spinal cord (left) or mouse ES cell-derived induced ureteric bud (right) and transplanted to immune-deficient mice. The left two panels shows the images of the kidney tissues at day 15 after transplantation. The punctate structures are glomeruli formed from metanephric mesenchyme. The number of glomeruli was counted to estimate the number of the formed nephrons and quantified in the right graph. SC: MM co-culture with fetal spinal cord, iUB: MM co-culture with induced ureteric bud.

### DETAILED DESCRIPTION OF EXEMPLARY EMBODIMENTS

The present disclosure will be described in detail referring to exemplary embodiments below, but the present disclosure is not limited to the embodiments described below. Unless otherwise specified in the text, all technical terms and scientific terms used in the present specification have the same meanings as generally understood by those skilled in the art to which the present disclosure belongs. Also, any materials and methods equivalent to or similar to those described in the present specification can be used in the practice of the present disclosure as well.

In addition, all publications and patents cited in the present specification in connection with the present disclosure described herein constitute a part of the present specification, for example, as those indicating methods, materials, etc. that can be used in the present disclosure.

In the present specification, “and/or” is used to include either one or both. In the present specification, “about” is used to mean that ±10% is allowed.

In the present specification, the term kidney organoid means a kidney-like tissue with a higher-order structure having a dendritic branching structure of a collecting duct that connects differentiated nephrons to each other, together with differentiated nephrons consisting of glomeruli and nephric tubules, and nephron progenitor cells and stromal cells.

The exemplary details of the exemplary embodiments of the present disclosure are described as follows.

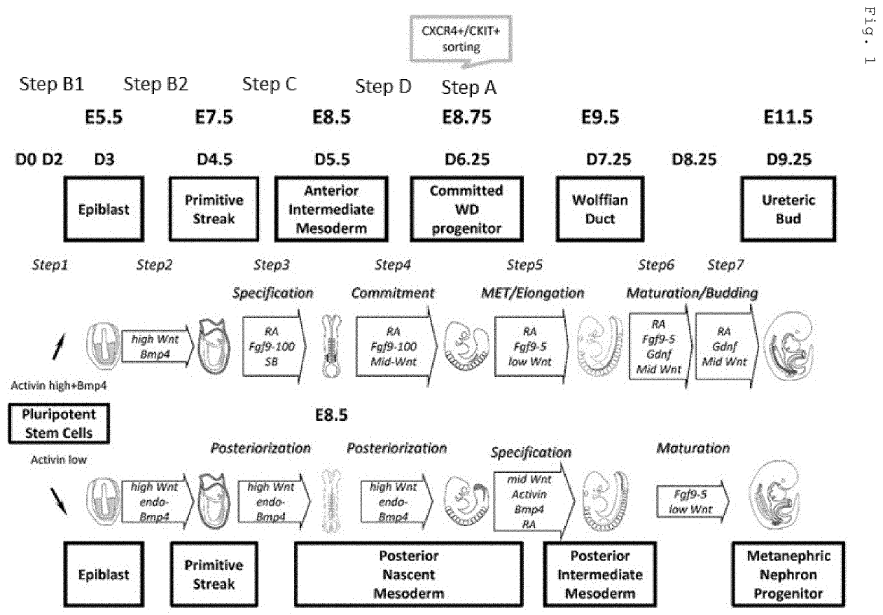

FIG. 1 illustrates an exemplary outline of the protocol for the production of higher-order structure of embryonic kidney, which employs differentially induced ureteric bud and nephron progenitor cells from pluripotent stem cells.

The upper panels show the exemplary embodiments of the present disclosure; the method of producing Wolffian duct (WD) progenitor-like cells and the following ureteric bud induction procedures. The lower panels show the outline of the protocol for inducing nephron progenitor cells. Procedures will be explained below.

The exemplary embodiment of the method of the present disclosure for producing Wolffian duct (WD) progenitor cell-like cells, comprising a procedure A (between procedure 4 and procedure 5 in the FIG. 1) of obtaining C-X-C chemokine receptor 4 (CXCR4)-positive and KIT proto-oncogene receptor tyrosine kinase (KIT)-positive cells

The exemplary embodiments of the present disclosure provide a method for producing Wolffian duct (WD) progenitor cell-like cells, comprising a procedure A of obtaining C-X-C chemokine receptor 4 (CXCR4)-positive and KIT proto-oncogene receptor tyrosine kinase (KIT)-positive cells (in the present specification, referred to also as a method 1 of the present disclosure).

When the WD progenitor-like cells produced by the method according to the exemplary embodiment(s) of the present disclosure is produced as a cell population, the cells are so produced that the proportion of CXCR4-positive and KIT-positive cells is 30% or more, preferably 50% or more, more preferably 70% or more, further preferably 80% or more, and more further preferably 90% or more, in all cells.

In the present specification, “WD progenitor-like cell” can mean, but is not limited to, e.g., a cell destined to differentiate into a ureteric bud cell having a branching ability with developmentally appropriate stimuli, and the WD progenitor-like cell is a cell expressing C-X-C chemokine receptor 4 (CXCR4) and KIT proto-oncogene receptor tyrosine kinase (KIT).

The developmentally appropriate stimulus means stimuli which differentiate WD progenitors into ureteric bud-like cells by a method according to the method described in examples. When the ureteric bud-like cells have a branching ability, it means that the ureteric buds formed by aggregation of the ureteric bud-like cells branch dendritically.

The WD progenitor-like cell further expresses preferably at least two, more preferably at least three, and further preferably all of Paired box (PAX) 2, LIM homeobox (LHX) 1, empty spiracles homeobox (EMX) 2, ret proto-oncogene (RET) and homeobox (HOX) B7, in addition to CXCR4 and KIT.

The WD progenitor-like cell is preferably a FLK1-negative (namely, vascular endothelial cell growth factor receptor 2 (VEGFR2)-negative) cell.

One example of the developmentally appropriate stimuli includes co-culturing nephron progenitor cells and an embryonic kidney-derived PDGFRa+ stromal cell population, by a method according to the method described in examples.

Cells (or cell population) that are positive for specific markers (for example, CXCR4, KIT, etc.) can be separated and obtained, for example, by using flow cytometry, i.e. FACS (fluorescence activated cell sorting), though the means is not limited to this. For example, regarding the CXCR4-positive cell, also a CXCR4-positive WD progenitor-like cell population can be separated, by a cell sorter, based on the binding strength to specific reagents such as an anti-CXCR4 antibody (for example, APC anti-human CD184 (CXCR4) Antibody, Clone 12G5, available from BioLegend, APC anti-mouse CD184 (CXCR4) Antibody, Clone L276F12, available from BioLegend) and the like and based on other parameters such as cell size and light scattering and the like. Further, for separation of the KIT-positive cell population, it is possible to use also an anti-KIT antibody (for example, PE anti-human CD117 (c-KIT) Antibody, Clone 104D2, available from BioLegend, CD117 (KIT) Monoclonal Antibody (2B8), PE, available from eBioscience) and the like.

Separation of cells (or cell population) that are positive for a marker can be carried out, for example, by FACS using an antibody specific to the marker and an isotype compatible control antibody. When the intensity of staining of cells with an antibody specific to a maker exceeds the intensity of staining of cells (or cell population) with an isotype compatible control antibody, the cells can be determined to be positive for the marker. When there is no difference between the intensity of staining of cells with an antibody specific to a maker and the intensity of staining of cells (or cell population) with an isotype compatible control antibody, the cells can be determined to be negative for the marker.

Further, cells which are positive for a specific marker can also be concentrated, depleted, separated, sorted and/or purified using conventional affinity or antibody techniques. For example, the separation of a specific cell type can be facilitated by binding labels, for example, magnetic beads; biotin that binds with high affinity to avidin or streptavidin; fluorescent dyes that can be used in fluorescent marking type cell separators; haptens; and equivalents, or the like to a ligand and/or an antibody.

In one exemplary embodiment of the present disclosure, the method according to the exemplary embodiment(s) of the present disclosure comprises a procedure of sorting CXCR4-positive and KIT-positive cells by a cell sorter.

Method for Inducing WD Progenitor-Like Cell from Pluripotent Stem Cell

Further, the present disclosure provides a method for inducing WD progenitor-like cells from pluripotent stem cells (referred to also as WD inducing method according to the exemplary embodiment(s) of the present disclosure).

Specifically, the present disclosure may include a method for inducing WD progenitor-like cells from pluripotent stem cells, comprising

(Procedure B1) a procedure of culturing pluripotent stem cells in a medium containing activin A or a tumor growth factor (Tgfb1 or Tgfb2),

(Procedure B2) a procedure of culturing cells obtained by procedure B1 in a medium containing a Wnt agonist (preferably GSK-3β inhibitor),

(Procedure C) a procedure of culturing cells obtained by procedure B2 in a medium containing retinoic acid (RA) or an RA analog (AGN193109, AM580, AM80, BMS453, BMS195614, AC 261066, AC55649, Isotretinoin), a fibroblast cell growth factor (FGF2, FGF4, FGF7, FGF9 or FGF20) and a TGFβ signal pathway inhibitor or a Wnt agonist (preferably GSK-3β inhibitor), and

(Procedure D) a procedure of culturing cells obtained by procedure C in a medium containing RA or an RA analog (AGN193109, AM580, AM80, BMS453, BMS195614, AC 261066, AC55649, Isotretinoin), a Wnt agonist (preferably GSK-3β inhibitor), and a fibroblast cell growth factor (FGF2, FGF4, FGF7, FGF9 or FGF20).

### Exemplary Procedure B1

In the present specification, “pluripotent stem cell (PSC)” may be any undifferentiated cell holding “self-replication” that enables proliferation while maintaining an undifferentiated state and “pluripotency” that allows differentiation into all three primary germ layers of the embryo. The pluripotent stem cells used in the present disclosure are preferably embryonic stem cells (ES) or induced pluripotent stem cells (iPS cell), and more preferably iPS cells.

ES cells are stem cells having pluripotency and proliferation ability based on self-replication, which can be established from the inner cell mass of an early embryo (for example, blastocyst). ES cells can be established by taking out the inner cell mass from the blastocyst of a fertilized egg and culturing the inner cell mass on fibroblast feeder cells. The methods of establishing and maintaining ES cells are known.

The induced pluripotent stem (iPS) cell is an artificial stem cell derived from a somatic cell, and can be produced by introducing a specific reprogramming factor in the form of DNA or protein into a somatic cell, and exhibits properties approximately equivalent to the ES cell (for example, differentiation pluripotency and proliferation ability based on self-replication) (K. Takahashi and S. Yamanaka (2006) Cell, 126: 663-676; K. Takahashi et al. (2007), Cell, 131: 861-872; J. Yu et al. (2007), Science, 318: 1917-1920; Nakagawa, M. et al., Nat. Biotechnol. 26: 101-106 (2008); WO2007/069666). The reprogramming factor may be constituted of a gene specifically expressed in the ES cell, its gene product or non-coding RNA thereof, a gene playing an important role in maintaining undifferentiated state of the ES cell, its gene product or non-cording RNA thereof, or a low molecular weight compound. Examples of the gene contained in the reprogramming factor include Oct3/4, Sox2, Sox1, Sox3, Sox15, Sox17, Klf4, Klf2, c-MYC, N-Myc, L-Myc, Nanog, Lin28, Fbx15, Eras, ECAT15-2, Tc11, beta-catenin, Lin28b, Sa111, Sa114, Esrrb, Nr5a2, Tbx3, Glis1 and the like. These reprogramming factors may be used singly or in combination. When the gene of c-MYC is introduced into a somatic cell and used as the reprogramming factor, it can be preferable to use an introduction method in which the introduced c-MYC gene is unlikely to be integrated into the chromosome of the target cell, after production of iPS cells, and the method includes, for example, but not limited to, introduction using a sendai virus vector or an episomal vector.

The method for producing ES cells or iPS cells, the method for culturing them, the method for maintaining an undifferentiated state thereof and the like are known per se, and ES cells or iPS cells can be produced and cultured according to methods described in the documents exemplified above or methods described in examples.

In the present disclosure, the culturing temperature is usually about 30 to 40° C., and preferably about 37° C., and the culture is conducted under an atmosphere of C02-containing air, and the C02 concentration is about 2 to 5%, and preferably 5%, though these conditions are not particularly restricted.

As the medium used in the present disclosure, a medium used for culturing animal cells can be prepared as a basal medium. Examples of the basal medium includes DMEM (Dulbecco's modified Eagle medium), DMEM/F12 medium, GMEM (Glasgow MEM) medium, Ham's F12 medium, IMDM (Iscove's modified Dulbecco's medium), αMEM (Eagle's minimum essential medium α modified type) and the like, and mixtures thereof, though they are not limited as long as desired cells can be obtained.

The medium used in the present disclosure may contain serum or may be serum-free. The medium used in the present disclosure may contain at least one or more medium additives such as, for example, albumin, N-2 supplement (Thermo Fisher Scientific), B-27 (registered trademark) supplement minus vitamin A (Thermo Fisher Scientific), 2-mercaptoethanol, 1-thioglycerol, amino acids, L-glutamine, non-essential amino acids, ascorbic acid and the like, if necessary.

Activin A means a homodimer of two inhibin BA chains, and in the present disclosure, it can be preferable to use a homodimer in which a Gly311-Ser426 fragment in which the N-terminal peptide of the inhibin BA chain (for example, NCBI accession number: NP_002183) is cleaved is disulfide-bonded, being an active type in which the N-terminal peptide is cleaved. Such activin A can be purchased from, for example, R&D Systems (R&D) and the like.

The concentration of activin A in a medium used in procedure B1 of the present disclosure (in the present specification, referred to also as medium B1) varies depending on cells to be used and the culturing time, the amount of a Wnt agonist used in procedure B2, and the like and is not particularly restricted as long as desired cells can be obtained, and is usually 1 ng/mL to 1000 ng/mL, preferably 1 ng/mL to 100 ng/mL, more preferably 3 ng/mL to 30 ng/mL, and further preferably about 10 ng/mL. In procedure B1, a tumor growth factor, Tgfb1 or Tgfb2 can be used instead of activin A. As the concentration of Tgfb1 or Tfgb2 to be used, the concentration of Tgfb1 or Tfgb2 providing the same effect, with reference to activin A, can be set.

The medium in procedure B1 may further contain an ROCK inhibitor. The ROCK inhibitor is not particularly restricted as long as it can suppress the function of a Rho kinase (ROCK), and in the present disclosure, for example, Y27632, Fasudil hydrochloride, GSK 429286, GSK 269962, AS 1892802, H 1152 dihydrochloride or HA 1100 hydrochloride can be used, and preferably Y27632 or Fasudil hydrochloride can be used, and more preferably Y27632 can be used. When Y27632 is used, the concentration is 1 μM to 1000 μM, preferably 1 μM to 100 μM, more preferably 1 μM to 100 μM, and further preferably about 10 μM. When another ROCK inhibitor is used instead of Y27632 in procedure B1, the concentration providing the same effect, with reference to Y27632, can be set.

The culturing time in procedure B1 is, for example, 5 days or less, preferably 0.5 to 3 days, and more preferably about 1 day.

In procedure B1, about 100 to 100,000 cells are aggregated to form an aggregate and suspension culture can be performed. The suspension culture can be performed, for example, using about 1,000 cells in the case of mouse and using about 10,000 cells in the case of human, though the number of cells is not limited to this.

The suspension culture is to culture cells in a non-adherent state to an incubator. The suspension culture can be performed by a method using one that has not been artificially treated for the purpose of improving adhesion to cells (for example, coating treatment with extracellular matrix, etc.), though the means is not particularly limited. Examples of such a cell non-adherent incubator include V-bottom 96-well low cell binding plate (Sumitomo Bakelite Co., Ltd.) and the like, though the incubator is not particularly restricted.

When human pluripotent stem cells (for example, human iPS cell) are used, the medium B1 preferably further contains a BMP signal pathway active substance (preferably BMP2, BMP4 or BMP7, more preferably BMP2 or BMP4, further preferably BMP4) from the standpoint of enhancing the proportion of both CXCR4 and KIT-positive cells in the cell population obtained by procedure D. The concentration of BMP4 in the medium B1 of the present disclosure varies depending on cells to be used and the culturing time, the amount of a BMP signal pathway active substance and the amount of a Wnt agonist used in procedure C and the like, and is not particularly restricted as long as desired cells can be obtained, and is, for example, 10 ng/mL or less, preferably 0.1 ng/mL to 10 ng/mL, more preferably 0.3 ng/mL to 3 ng/mL, and further preferably about 1 ng/mL. When another BMP signal pathway active substance is used, the concentration at which it is possible to exhibit the same effect as that obtained in the case of use of BMP4 can be appropriately selected.

When mouse pluripotent stem cells (for example, mouse ES cell) are used, it can be preferable that the cells disaggregated by Accutase (ESGRO) or the like are aggregated at a proportion of about 1,000 cells per aggregate, then, cultured for about 2 days in a medium having the same components as in the medium B1 of the present disclosure excepting that no activin A is contained, and the resultant culture is subjected to procedure B1 of the present disclosure.

### Exemplary Procedure B2

Immature mesoderm cells can be obtained by culturing the result of the culture obtained by the culture in procedure B1 in a medium containing a Wnt agonist (preferably a GSK-3β inhibitor).

The Wnt agonist is defined as an agent that activates TCF/LEF-mediated transcription in cells. The Wnt agonist is therefore selected from true Wnt agonists that bind to and activate Frizzled receptor family members, including all kinds of Wnt family proteins, and inhibitors for intracellular β-catenin degradation and activators for TCF/LEF. The Wnt agonist also includes Wnt signal pathway inhibitors, GSK-3β inhibitors, Dkk1 antagonists and the like.

The GSK-3β inhibitor is defined as a substance that inhibits the kinase activity of the Glycogen Synthase Kinase (GSK)-33 protein (e.g., phosphorylation ability to β-catenin), and many substances such as, for example, CHIR99021 (CAS No: 252917-06-9), BIO (CAS number: 667463-62-9), SB216763 (CAS number: 280744-9-4) and the like are already known.

The Wnt agonist used in the present disclosure is preferably CHIR99021, SB216763, BIO, A 1070722, Lithium carbonate, 3F8, SB 415286, TDZD 8, TWS 119, TCS 2002, Wnt3 or Wnt3a, more preferably CHIR99021 or SB216763, and further preferably CHIR99021.

When CHIR99021 can be used as the Wnt agonist in procedure B2, the concentration of CHIR99021 in the medium used in procedure B2 (also referred to as medium B2 in the present specification) varies depending on cells to be used and the culturing time, the amount of activin A used in procedure B1 and the like, and is not particularly limited as long as desired cells can be obtained, and is usually 1 μM to 1000 μM, preferably 1 μM to 200 μM, more preferably 3 μM to 30 μM, and further preferably about 10 μM. When another Wnt agonist is used instead of CHIR99021 in procedure B2, the concentration providing the same effect, with reference to CHIR99021, can be set.

The culturing time in procedure B2 is preferably about 1 to 2 days, and more preferably about 1.5 days from the standpoint of enhancing the proportion of both CXCR4 and KIT-positive cells in the cell population obtained in procedure D.

Procedure B2 can be performed, for example, by replacing the medium B1 with the medium B2 in procedure 2 after the culture in procedure B1.

The medium B2 can further contain a BMP signal pathway active substance (preferably BMP2, BMP4 or BMP7, more preferably BMP2 or BMP4, further preferably BMP4). The concentration of a BMP signal pathway active substance contained in the medium B2 can be appropriately adjusted to enhance the proportion of both CXCR4 and KIT-positive WD progenitor-like cells in the cell population obtained by procedure D, according to the amount of activin A or the like used in procedure B1, and it is, for example, 10 ng/mL or less, preferably 5 ng/mL or less, and more preferably 0.3 ng/mL to 3 ng/mL. When another BMP signal pathway active substance is used, the concentration at which it is possible to exhibit the same effect as that obtained in the case of use of BMP4 can be appropriately selected.

### Exemplary Procedure C

In one embodiment of the present disclosure, the result of the culture obtained by the culture of procedure B2 is cultured in a medium containing RA or an RA analog, a fibroblast cell growth factor (FGF2, Fgf4, Fgf7, FGF9 or FGF20), and a TGFβ signal pathway inhibitor or a Wnt agonist (preferably GSK-3β inhibitor).

Examples of the retinoic acid that can be used in the present disclosure include all trans-retinoic acid (ATRA), which can be purchased from Sigma-Aldrich and the like. In addition, retinoic acid artificially modified while retaining the functions of natural retinoic acid can also be used.

When ATRA is used as the retinoic acid in procedure C, the concentration of ATRA in the medium C varies depending on the culture conditions and the like, and is not particularly limited as long as desired cells can be obtained, and is usually 10 nM to 1 μM, preferably 10 to 500 nM, more preferably 50 nM to 200 nM, and further preferably about 100 nM.

In procedure C, a retinoic acid analog can be used instead of retinoic acid. Examples of the retinoic acid analog include AGN193109, AM580, AM80, BMS453, BMS195614, AC 261066, AC55649 and Isotretinoin, and AGN193109 is particularly preferable. When an RA analog is used instead of RA in procedure C, the concentration providing the same effect, with reference to RA, can be set.

The fibroblast cell growth factor (FGF2, FG4, FGF7, FGF9, or FGF20) used in the medium C may be prepared by referring to a method known per se based on known amino acid sequence information, or a recombinant human FGF protein purchased from R & D Systems or the like can also be used. The FGF used is preferably FGF9 or FGF20, and more preferably FGF9. The concentration of FGF9 protein in the medium C varies depending on the culture conditions and the like, and is, for example, 10 ng to 1 μg/mL, preferably 10 ng to 500 ng/mL, more preferably 50 ng to 200 ng/mL, and further preferably about 100 ng/mL. When another FGF is used instead of FGF9 in procedure C, the concentration providing the same effect, with reference to FGF9, can be set.

The TGFβ signal pathway inhibitor is defined as a substance that inhibits signal transduction continuing from binding to a receptor of TGFβ to SMAD, and for example, many substances have been reported, such as a substance that inhibits the binding to the ALK family, which is a receptor for TGFβ, and a substance that inhibits the phosphorylation of SMAD by the ALK family.

The TGFβ signal pathway inhibitor or Wnt agonist used in procedure C of the present disclosure is not particularly limited as long as desired cells can be obtained in procedure D, and includes ALK inhibitors: SB431542 (CAS No: 301836-41-9) or A83-01 (CAS No.: 909910-43-6), D4476, GW788388, LY364947, R268712, RepSox, SB505124, SB525334 or SD208, and SB431542 or A83-01 can be preferable, and SB431542 is more preferred.

When SB431542 is used as the TGFβ signal pathway inhibitor or Wnt agonist in procedure C, the concentration of SB431542 in the medium used in procedure C (also referred to as medium C in the present specification) varies depending on the culture conditions and the like, and is not particularly limited as long as desired cells can be obtained, and is usually 1 μM to 1000 μM, preferably 3 μM to 500 μM, and more preferably 10 μM to 200 μM, and when mouse pluripotent stem cells are used in procedure B1, it is more preferably about 10 μM, while when human pluripotent stem cells are used in procedure B1, it is more preferably about 100 μM. When another component is used instead of SB431542 in procedure C, the concentration providing the same effect, with reference to SB431542, can be set.

The medium C may be substantially free of GSK-3β inhibitors. The concentration thereof is not particularly limited as long as desired cells can be obtained, and specifically, it is preferably about 5 μM or less, and preferably about 3 μM or less.

The culturing time in procedure C is not particularly limited as long as the proportion of WD progenitor-like cells in the cell population obtained in procedure D is not reduced, and is, for example, about 1 to 3 days, and more preferably about 1 to 2 days.

Procedure C can be performed, for example, by replacing the medium B2 with the medium C after culturing in procedure B2.

When human pluripotent stem cells (preferably human iPS cells) are used in procedure B1, the medium C may further contain a BMP signal pathway inhibitor (preferably LDN193189, Noggin, Gremlin, DMH-1, DMH2, Dorsomorphin dihydrochloride, K 02288, LDN 212854, or ML 347 can be mentioned, more preferably LDN 193189 or Noggin, still more preferably LDN 193189 can be mentioned).

When mouse pluripotent stem cells (preferably mouse ES cells) are used in procedure B1, it can be preferable that the medium C does not contain either a BMP signal pathway inhibitor or a BMP signal pathway active substance.

When the medium C contains LDN193189, the concentration of LDN193189 in the medium C is preferably 1 nM to 1000 nM, more preferably 3 nM to 500 nM, further preferably 10 nM to 200 nM, and still further preferably about 100 nM. When another substance is used instead of LDN193189 in procedure C, the concentration providing the same effect, with reference to LDN193189, can be set.

### Exemplary Procedure D

In one exemplary embodiment of the present disclosure, the result of the culture obtained by the culture in procedure C is cultured in the medium D containing RA or an RA analog, a Wnt agonist (preferably GSK-3β inhibitor) and a fibroblast cell growth factor (FGF2, FGF4, FGF7, FGF9 or FGF20).

When ATRA is used as the retinoic acid in procedure D, the concentration of ATRA in the medium used in procedure D (also referred to as medium D in the present specification) varies depending on the culture conditions and the like, and is not particularly limited as long as desired cells can be obtained, and is usually 10 nM to 1 μM, preferably 10 nM to 500 nM, more preferably 50 nM to 200 nM, and further preferably about 100 nM.

In procedure D, a retinoic acid analog can be used instead of retinoic acid. As the retinoic acid analog, the compounds listed in procedure C can be mentioned, and AGN193109 can be preferable. When an RA analog is used instead of RA in procedure D, the concentration providing the same effect, with reference to RA, can be set.

The FGF that can be used in the medium D is the same as in procedure C, and FGF9 or FGF20 can be preferable, and FGF9 is more preferable. The concentration of FGF9 protein in the medium D varies depending on the culture conditions and the like, and is, for example, 10 ng to 1 μg/mL, preferably 10 ng to 500 ng/mL, more preferably 30 ng to 300 ng/mL, and further preferably about 100 ng/mL. When another FGF is used instead of FGF9 in procedure D, the concentration providing the same effect, with reference to FGF9, can be set.

As the Wnt agonist used in procedure D, the components listed in procedure B2 can be used, and CHIR99021 or SB216763 can be preferable, and CHIR99021 is more preferable. When CHIR99021 is used in procedure D, the concentration of CHIR99021 in the medium D is not particularly limited as long as desired cells are obtained, and is usually 0.1 μM to 100 μM, preferably 1 μM to 100 μM, more preferably 1 μM to 10 μM, and further preferably about 3 μM to about 5 μM. When another Wnt agonist is used instead of CHIR99021 in procedure D, the concentration providing the same effect, with reference to CHIR99021, can be set.

The culturing time in procedure D is not particularly limited as long as the proportion of WD progenitor-like cells in the cell population obtained in procedure D does not decrease, and is, for example, about 1 to 3 days, and preferably about 1.5 to 2.5 days.

Procedure D can be performed, for example, by replacing the medium C with the medium D after culturing in procedure C.

When human pluripotent stem cells (preferably human iPS cells) are used in procedure B1, the medium D may further contain a BMP signal pathway inhibitor. Examples of the BMP signal pathway inhibitor that can be used in procedure D include the substances listed in procedure C, and LDN193189 or Noggin can be preferable, and LDN193189 is more preferable. When the medium D contains LDN193189, the concentration of LDN193189 in the medium D is preferably 1 nM to 500 nM, more preferably 10 nM to 100 nM, further preferably 10 nM to 50 nM, and still further preferably about 30 nM. When another substance is used instead of LDN193189 in procedure D, the concentration providing the same effect, with reference to LDN193189, can be set.

As the result of the culture of procedure D, a cell population containing WD progenitor-like cells can be obtained.

The exemplary production method of WD progenitor-like cells of the present disclosure is preferably

a method for producing Wolffian duct (WD) progenitor cell-like cells comprising a procedure A of obtaining CXCR4-positive and KIT-positive cells (preferably, further Pax2, Lhx1, Emx2, RET and HOXB7-positive cells, more preferably Flk1-negative cells, in addition to CXCR4-positive and KIT-positive cells), wherein the method comprises the following procedures B1, B2, C and D:

(Procedure B1) a procedure of culturing pluripotent stem cells (preferably ES cells or iPS cells, more preferably human iPS cells or mouse ES cells) in a medium B1 containing activin A (1 ng/mL to 1000 ng/mL, preferably 1 ng/mL to 100 ng/mL, more preferably 3 to 30 ng/mL of activin A) (in culturing with human iPS cells, containing preferably 10 ng/mL or less, preferably 0.1 ng/mL to 10 ng/mL, more preferably 0.3 ng/mL to 3 ng/mL, and further preferably about 1 ng/mL of BMP4, or a BMP signal pathway active substance having the concentration at which it is possible to exhibit the same effect, in addition to activin A) (preferably, suspension culture is conducted; and the culturing period is 5 days or less, preferably 0.5 to 3 days, and more preferably about 1 day),

(Procedure B2) a procedure of culturing cells obtained by procedure B1 in a medium B2 containing a Wnt agonist, preferably a GSK-3β inhibitor (preferably CHIR99021, further 1 μM to 1000 μM, preferably 1 μM to 200 μM, more preferably 3 μM to 30 μM, and further preferably about 10 μM of CHIR99021) (preferably, containing 10 ng/mL or less, preferably 5 ng/mL or less, and more preferably 0.3 ng/mL to 3 ng/mL of BMP4, or a BMP signal pathway active substance having the concentration at which it is possible to obtain the same effect, in addition to the Wnt agonist) (preferably, suspension culture is conducted; and the culturing period is preferably about 1 to 2 days, and more preferably about 1.5 days),

(Procedure C) a procedure of culturing cells obtained by procedure B2 in a medium C containing retinoic acid (preferably ATRA, further 10 nM to 1 μM, preferably 10 to 500 nM, more preferably 50 nM to 200 nM, and further preferably about 100 nM of ATRA), FGF9 (10 ng to 1 μg/mL, preferably 10 ng to 500 ng/mL, more preferably 50 ng to 200 ng/mL, and further preferably about 100 ng/mL of FGF9) and a TGFβ signal pathway inhibitor (preferably SB431542, further 1 μM to 1000 μM, preferably 3 μM to 500 μM, and more preferably 10 μM to 200 μM of SB431542, and when mouse pluripotent stem cells are used in procedure B1, about 10 μM of SB431542, while when human pluripotent stem cells are used in procedure B1, about 100 μM of SB431542) (when human pluripotent stem cells (preferably human iPS cells) are used in procedure B1, further a BMP signal pathway inhibitor (preferably LDN193189, containing 1 nM to 1000 nM, preferably 3 nM to 500 nM, more preferably 10 nM to 200 nM, and further preferably about 100 nM of LDN193189) (culturing for preferably about 1 to 2 days, and more preferably about 1 day), and

(Procedure D) a procedure of culturing cells obtained by procedure C in a medium D containing RA (preferably ATRA, further 10 nM to 1 μM, preferably 10 to 500 nM, more preferably 50 nM to 200 nM, and further preferably about 100 nM of ATRA), FGF9 (10 ng to 1 μg/mL, preferably 10 ng to 500 ng/mL, more preferably 30 ng to 300 ng/mL, and further preferably about 100 ng/mL of FGF9), and a GSK-3β inhibitor (preferably CHIR99021, further 0.1 μM to 100 μM, preferably 1 μM to 100 μM, more preferably 1 μM to 10 μM, and further preferably about 3 μM to 5 μM of CHIR99021) (when human pluripotent stem cells (preferably human iPS cells) are used in procedure B1, further containing a BMP signal pathway inhibitor (preferably LDN193189, containing 1 nM to 500 nM, preferably 10 nM to 100 nM, more preferably 10 nM to 50 nM, and further preferably about 30 nM of LDN193189) (culturing for preferably about 1 to 3 days, more preferably about 1.5 days to 2.5 days).

Further, the present disclosure provides a kit for producing WD progenitor-like cells from pluripotent stem cells, with any one or more mediums selected from the group consisting of the mediums A, B, C and D, or two or more mediums, more preferably three or more mediums, and further preferably four mediums selected from the group in combination.

The medium can be provided as a liquid medium or a powder medium, or can be provided as a medium additive that can provide the medium of the present disclosure by adding to a commercially available basal medium.

The kit may include an anti-CXCR4 antibody and/or an anti-KIT antibody in addition to the medium or the medium additives described above. The antibody may be provided in the form bound to a labeled molecule, such as a fluorescent molecule, for cell sorting.

Further, the present disclosure provides a kit for producing ureteric bud-like cells from pluripotent stem cells, further comprising any one or more mediums selected from the group consisting of mediums E, F and G described later, or two or more mediums, more preferably three mediums selected from the group, in addition to the kit described above.

### Exemplary Procedure E

The WD progenitor-like cells of the present disclosure can be subjected to further maturation culture and differentiated into ureteric bud-like cells. The present disclosure provides a method for producing ureteric bud-like cells, further comprising a procedure E of culturing CXCR4-positive and KIT-positive WD progenitor-like cells in a medium containing RA or an RA analog, a Wnt agonist (preferably a GSK-3β inhibitor or Rspondin1), a fibroblast cell growth factor (FGF2, FGF4, FGF7, FGF9 or FGF20), and an ROCK inhibitor (in the present specification, also referred to as medium E)

When ATRA is used as the retinoic acid in procedure E, the concentration of ATRA in the medium E varies depending on the culture conditions and the like, and is not particularly limited as long as desired cells can be obtained, and is usually 10 nM to 1 μM, preferably 10 nM to 500 nM, more preferably 50 nM to 200 nM, and further preferably about 100 nM.

In procedure E, a retinoic acid analog can be used instead of retinoic acid. As the retinoic acid analog, the compounds listed in procedure C can be mentioned, and AGN193109 can be preferable. When an RA analog is used instead of RA in procedure E, the concentration providing the same effect, with reference to RA, can be set.

The fibroblast cell growth factor that can be used in the medium E is the same as in procedure C, and FGF9 or FGF20 can be preferable, and FGF9 is more preferable. The concentration of the FGF9 protein in the medium E is not particularly restricted as long as desired ureteric bud-like cells can be obtained, and is, for example, 0.1 ng/mL to 100 ng/mL, preferably 0.5 ng/mL to 50 ng/mL, more preferably 2 ng/mL to 10 ng/mL, and further preferably about 5 ng/mL. When another FGF is used instead of FGF9 in procedure E, the concentration providing the same effect, with reference to FGF9, can be set.

As the Wnt agonist used in procedure E, the components listed in procedure B2 and, additionally, Rspondin1 can be used, and CHIR99021, SB216763 or Rspondin1 can be preferable, and CHIR99021 is more preferable. When CHIR99021 is used as the Wnt agonist in procedure E, the concentration of CHIR99021 in the medium E is not particularly limited as long as desired cells can be obtained, and is, for example, 0.1 μM to 100 μM, preferably 0.1 μM to 10 μM, more preferably 0.3 μM to 5 μM, and further preferably about 1 μM. When another Wnt agonist is used instead of CHIR99021 in procedure E, the concentration providing the same effect, with reference to CHIR99021, can be set.

The medium E may further contain an ROCK inhibitor from the standpoint of increasing the survival rate of cells. The ROCK inhibitor is not particularly limited as long as it can suppress the function of Rho kinase (ROCK), and specific examples thereof include the substances listed in procedure B1, and Y27632 or Fasudil hydrochloride can be preferable, and Y27632 is more preferable. When Y27632 is used, the concentration thereof is 1 μM to 1000 μM, preferably 1 μM to 100 μM, more preferably 1 μM to 50 μM, and further preferably about 10 μM. When another Rock inhibitor is used instead of Y27632 in procedure E, the concentration providing the same effect, with reference to Y27632, can be set.

In a preferred exemplary embodiment, the medium E may further contain a support for the culture, including, for example, but not limited to, growth factor reduced Matrigel, collagen and laminin. When the medium E contains a growth factor reduced Matrigel, its concentration in the medium E is, for example, 5% to 50%, preferably 5% to 20%, more preferably 10% to 20%, and further preferably about 10%.

When the WD progenitor-like cells are induced from human pluripotent stem cells, the medium E may contain yet another fibroblast cell growth factor (FGF1, FGF2, FGF4, FGF5, FGF6, FGF7, FGF10 or FGF20, preferably FGF1 or FGF2, and more preferably FGF1) in addition to the FGF described above.

The FGF (for example, FGF1) used for the medium E may be prepared by referring to a method known per se based on known amino acid sequence information, or a recombinant human FGF protein purchased from R & D Systems or the like can also be used. The concentration of the FGF1 protein in the medium E varies depending on the culture conditions and the like, and is, for example, 10 ng to 1 μg/mL, preferably 10 ng to 500 ng/mL, more preferably 50 ng to 200 ng/mL, and further preferably about 100 ng/mL. When another FGF is used instead of FGF1 in procedure E, the concentration providing the same effect, with reference to FGF1, can be set.

The combination of FGF essential for procedure E and FGF for culturing WD progenitor-like cells induced from human pluripotent stem cells includes, but not limited to, for example, combinations of FGF9 or FGF20 with FGF1 or FGF2, preferably a combination of FGF9 and FGF1.

If the WD progenitor-like cells are induced from human pluripotent stem cells, the medium E may further contain a BMP signal pathway inhibitor. Examples of the BMP signal pathway inhibitor that can be used in procedure E include the substances listed in procedure C, preferably LDN193189 or Noggin, and more preferably LDN193189.

When the medium E contains LDN 193189, the concentration of the medium is preferably 1 nM to 300 nM, more preferably 1 nM to 100 nM, further preferably 1 nM to 20 nM, and more further preferably about 10 nM. When another substance is used instead of LDN193189 in procedure E, the concentration providing the same effect, with reference to LDN193189, can be set.

The culturing time in procedure E is not particularly limited, and is, for example, about 1 to 5 days, and more preferably about 1 to 3 days, and when the WD progenitor-like cells are induced from human pluripotent stem cells, it is more preferably about 2 to 3 days.

In procedure E, WD precursor cell-like cells composed of about 100 to 100,000 cells (for example, about 10,000 cells) can be aggregated to form an aggregate and suspension culture can be performed, and as the incubator, a V-bottom 96-well low cell binding plate (Sumitomo Bakelite) or the like can be used, though the incubator is not particularly restricted.

A procedure of culturing CXCR4-positive and KIT-positive WD progenitor-like cells (preferably WD progenitor-like cells produced by procedures B1, B2, C, D and A; preferably 100 to 100,000 cells, and more preferably 1000 to 50,000 cells) in a medium E containing retinoic acid (preferably ATRA, and 10 nM to 1 μM, preferably 10 nM to 500 nM, more preferably 50 nM to 200 nM, and further preferably about 100 nM of ATRA), a Wnt agonist, preferably a GSK-3β inhibitor or Rspondin1 (preferably CHIR99021, for example, 0.1 μM to 100 μM, preferably 0.1 μM to 10 μM, more preferably 0.3 μM to 5 μM, and further preferably about 1 μM of CHIR99021), FGF9 (0.1 ng/mL to 100 ng/mL, preferably 0.5 ng/mL to 50 ng/mL, more preferably 2 ng/mL to 10 ng/mL, and further preferably about 5 ng/mL of FGF9) and an ROCK inhibitor (preferably Y27632, and 1 μM to 1000 μM, preferably 1 μM to 100 μM, more preferably 1 μM to 50 μM, and further preferably about 10 μM of Y27632) (preferably further containing a growth factor reduced Matrigel, and containing 5% to 50%, preferably 5% to 20%, more preferably 10% to 20%, and further preferably about 10% of a growth factor reduced Matrigel) (preferably suspension aggregate culture is performed, more preferably culturing as a suspension aggregate on a low adherent incubator; preferably culturing for about 1 to 5 days, and when the WD progenitor-like cells are induced from human pluripotent stem cells, culturing for about 2 to 3 days, while when derived from mouse pluripotent stem cells, culturing for about 1 to 3 days).

In a preferred exemplary embodiment, the cells obtained in procedure E are Emx2 and Ret-positive and Hnf1b, Wnt9b, Calb1 and E-cadherin-positive.

### Exemplary Procedure F

The exemplary embodiments of the present disclosure can provide a method for producing ureteric bud-like cells, further comprising a procedure (F) of culturing cells obtained by procedure (E) in a medium containing RA or an RA analog, a Wnt agonist (preferably a GSK-3β inhibitor or Rspondin1), a fibroblast cell growth factor (FGF2, FGF4, FGF7, FGF9 or FGF20), an ROCK inhibitor, and a glial cell line-derived neurotrophic factor (GDNF) or a GDNF analog (BT18 or SIB4035) or FGF10 (in the present specification, also referred to as medium F).

When ATRA is used as the retinoic acid in procedure F, the concentration of ATRA in the medium varies depending on the culture conditions and the like, and is not particularly limited as long as desired cells can be obtained, and is usually 10 nM to 1 μM, preferably 10 nM to 500 nM, more preferably 50 nM to 200 nM, and further preferably about 100 nM.

In procedure F, a retinoic acid analog can be used instead of retinoic acid. As the retinoic acid analog, the compounds listed in procedure C can be mentioned, and AGN193109 can be preferable. When an RA analog is used instead of RA in procedure F, the concentration providing the same effect, with reference to RA, can be set.

The fibroblast cell growth factor that can be used in the medium F is the same as in procedure C, and FGF9 or FGF20 can be preferable, and FGF9 is more preferable. The concentration of the FGF9 protein in the medium F is not particularly restricted as long as desired ureteric bud-like cells can be obtained, and is, for example, 0.1 ng to 100 ng/mL, preferably 0.5 ng to 50 ng/mL, more preferably 2 ng to 10 ng/mL, and further preferably about 5 ng/mL. When another FGF is used instead of FGF9 in procedure F, the concentration providing the same effect, with reference to FGF9, can be set.

As the Wnt agonist that can be used in procedure F, the components listed in procedure B2 and, additionally, Rspondin1 can be used, and CHIR99021, SB216763 or Rspondin1 can be preferable, and CHIR99021 is more preferable. When CHIR99021 is used as the Wnt agonist in procedure F, the concentration of CHIR99021 in the medium F is not particularly limited as long as desired cells can be obtained, and is usually 0.1 μM to 300 μM, preferably 0.3 μM to 100 μM, more preferably 1 μM to 5 μM, and further preferably about 3 μM. When another Wnt agonist is used instead of CHIR99021 in procedure F, the concentration providing the same effect, with reference to CHIR99021, can be set.

The glial cell line-derived neurotrophic factor (GDNF) used in the medium F may be prepared by referring to a method known per se based on known amino acid sequence information, and a recombinant human GDNF protein purchased from R & D Systems or the like can also be used. The concentration of the GDNF protein in the medium F varies depending on the culture conditions and the like, and is, for example, 0.1 ng to 100 ng/mL, preferably 0.1 ng to 10 ng/mL, more preferably 0.5 ng to 10 ng/mL, and further preferably about 1 ng/mL.

In the medium F, a GDNF analog or FGF10 can be used instead of GDNF. As the GDNF analog, BT18 or SIB4035 can be mentioned, and BT18 can be preferable. When a GDNF analog or FGF10 is used instead of GDNF in procedure F, the concentration providing the same effect, with reference to GDNF, can be set.

The medium F may further contain an ROCK inhibitor from the standpoint of increasing the survival rate of cells. The ROCK inhibitor is not particularly limited as long as it can suppress the function of Rho kinase (ROCK), and specific examples thereof include the substances listed in procedure B1, preferably Y27632 or Fasudil hydrochloride, and more preferably Y27632. When the medium F contains Y27632, the concentration in the medium is, for example, 1 μM to 1000 μM, preferably 1 μM to 100 μM, more preferably 1 μM to 50 μM, and further preferably about 10 μM. When another Rock inhibitor is used instead of Y27632 in the medium F, the concentration providing the same effect, with reference to Y27632, can be set.

In a preferred exemplary embodiment, the medium F may further contain a growth factor reduced Matrigel. When the medium F contains a growth factor reduced Matrigel, its concentration in the medium F is, for example, 5% to 50%, preferably 5% to 20%, more preferably 10% to 20%, and further preferably about 10%.

When the WD progenitor-like cells are induced from human pluripotent stem cells, the medium F may contain still another fibroblast cell growth factor (FGF1, FGF2, FGF4, FGF5, FGF6, FGF7, FGF10 or FGF20, preferably FGF1 or FGF2, more preferably FGF1) in addition to the FGF described above.

The FGF (for example, FGF1) used for the medium F may be prepared by referring to a method known per se based on known amino acid sequence information, or a recombinant human FGF protein purchased from R & D Systems or the like can also be used. The concentration of the FGF1 protein in the medium F varies depending on the culture conditions and the like, and is, for example, 10 ng to 1 μg/mL, preferably 10 ng to 500 ng/mL, more preferably 50 ng to 200 ng/mL, and further preferably about 100 ng/mL. When another FGF is used instead of FGF1 in procedure F, the concentration providing the same effect, with reference to FGF1, can be set.

The combination of FGF essential for procedure F and FGF for culturing WD progenitor-like cells induced from human pluripotent stem cells includes, for example, combinations of FGF9 or FGF20 with FGF1 or FGF2, and a combination of FGF9 and FGF1 is preferred, thought the combination is not limited to this.

When the WD progenitor-like cells are induced from human pluripotent stem cells, the medium F may further contain a BMP signal pathway inhibitor. Examples of the BMP signal pathway inhibitor that can be used in procedure F include the substances listed in procedure C, preferably LDN193189 or Noggin, and more preferably LDN193189. When the medium F contains LDN193189, the concentration thereof in the medium is preferably 1 nM to 300 nM, more preferably 1 nM to 100 nM, further preferably 5 nM to 20 nM, and still further preferably about 10 nM. When another substance is used instead of LDN193189 in procedure F, the concentration providing the same effect, with reference to LDN193189, can be set.

The culturing time in procedure F is not particularly limited, and is, for example, about 1 to 5 days, and more preferably about 1 to 3 days. More preferably, when the WD progenitor-like cells used in procedure (E) are induced from mouse pluripotent stem cells, it is about 1 day, while when the WD progenitor-like cells used in procedure (E) are induced from human pluripotent stem cells, it is about 2 days.

In a preferred exemplary embodiment, procedure F is a procedure of culturing cells obtained by procedure (E) (preferably cell aggregate) in a medium containing retinoic acid (preferably ATRA, and 10 nM to 1 μM, preferably 10 nM to 500 nM, more preferably 50 nM to 200 nM, and further preferably about 100 nM of ATRA), a Wnt agonist (preferably CHIR99021, and 0.1 μM to 300 μM, preferably 0.3 μM to 100 μM, more preferably 1 μM to 5 μM, and further preferably about 3 μM of CHIR99021), an ROCK inhibitor (preferably Y27632, and 1 μM to 1000 μM, preferably 1 μM to 100 μM, more preferably 1 μM to 50 μM, and further preferably about 10 μM of Y27632) and GDNF (0.1 ng to 100 ng/mL, preferably 0.1 ng to 10 ng/mL, more preferably 0.5 ng to 10 ng/mL, and further preferably about 1 ng/mL of GDNF) (preferably further containing a growth factor reduced Matrigel, and containing 5% to 50%, preferably 5% to 20%, more preferably 10% to 20%, and further preferably about 10% of growth factor reduced Matrigel; and when the WD progenitor-like cells used in procedure (E) are induced from human pluripotent stem cells, further containing preferably 10 ng to 1 μg/mL, more preferably 10 ng to 500 ng/mL, further preferably 50 ng to 200 ng/mL, and more further preferably about 100 ng/mL of FGF1; while when the WD progenitor-like cells used in procedure (E) are induced from human pluripotent stem cells, further containing preferably 1 nM to 300 nM, more preferably 1 nM to 100 nM, further preferably 5 nM to 20 nM, and more further preferably about 10 nM of LDN193189) (preferably suspension aggregate culture is performed, more preferably culturing as a suspension aggregate on a low adherent incubator; preferably culturing for about 1 to 5 days, more preferably culturing for about 1 to 3 days).

### Exemplary Procedure G

The exemplary embodiments of the present disclosure provide a method for producing ureteric bud-like cells, further comprising (Procedure G) a procedure of culturing cells obtained by procedure F in a medium containing RA or an RA analog, a Wnt agonist (preferably a GSK-3β inhibitor or Rspondin1), an ROCK inhibitor, and GDNF or a GDNF analog (BT18 or SIB4035) or FGF10 (in the present specification, also referred to as medium G).

When ATRA is used as the retinoic acid in procedure G, the concentration of ATRA in the medium varies depending on the culture conditions and the like, and is not particularly limited as long as desired cells can be obtained, and is usually 10 nM to 1 μM, preferably 10 nM to 500 nM, more preferably 50 nM to 200 nM, and further preferably about 100 nM.

In procedure G, a retinoic acid analog can be used instead of retinoic acid. As the retinoic acid analog, the compounds listed in procedure C can be mentioned, and AGN193109 can be preferable. When an RA analog is used instead of RA in procedure E, the concentration providing the same effect, with reference to RA, can be set.

As the Wnt agonist that can be used in procedure G, the components listed in procedure B2 and, additionally, Rspondin1 can be used, and CHIR99021, SB216763 or Rspondin1 can be preferable, and CHIR99021 is more preferable. When CHIR99021 is used as the Wnt agonist in procedure G, the concentration of CHIR99021 in the medium G is not particularly limited as long as desired cells can be obtained, and is usually 0.1 μM to 300 μM, preferably 0.3 μM to 100 μM, more preferably 1 μM to 5 μM, and further preferably about 3 μM. When another Wnt agonist is used instead of CHIR99021 in procedure G, the concentration providing the same effect, with reference to CHIR99021, can be set.

The concentration of the GDNF protein in the medium G is not particularly restricted as long as desired ureteric bud-like cells can be obtained, and is, for example, 0.1 ng to 100 ng/mL, preferably 0.2 ng to 20 ng/mL, more preferably 0.5 ng to 10 ng/mL, and further preferably about 2 ng/mL.

In the medium G, a GDNF analog or FGF10 can be used instead of GDNF. As the GDNF analog, BT18 or SIB4035 can be mentioned, and BT18 can be preferable. When a GDNF analog or FGF10 is used instead of GDNF in procedure G, the concentration providing the same effect, with reference to GDNF, can be set.

The medium G may further contain an ROCK inhibitor from the standpoint of enhancing the survival rate of cells. The ROCK inhibitor is not particularly limited as long as it can suppress the function of Rho kinase (ROCK), and specific examples thereof include the substances listed in procedure B1, preferably Y27632 or Fasudil hydrochloride, and more preferably Y27632. When the medium G contains Y27632, the concentration thereof in the medium is 1 μM to 1000 μM, preferably 1 μM to 100 μM, more preferably 1 μM to 50 μM, and further preferably about 10 μM. When another Rock inhibitor is used instead of Y27632 in the medium G, the concentration providing the same effect, with reference to Y27632, can be set.

In a preferred exemplary embodiment, the medium G may further contain a growth factor reduced Matrigel. When the medium G contains a growth factor reduced Matrigel, its concentration in the medium G is, for example, 5% to 50%, preferably 5% to 20%, more preferably 10% to 20%, and further preferably about 10%.

When the WD progenitor-like cells used in procedure (E) are induced from human pluripotent stem cells, the medium G may further contain a fibroblast cell growth factor (FGF1, FGF2, FGF4, FGF5, FGF6, FGF7, FGF10 or FGF20, preferably FGF1 or FGF2, and more preferably FGF1). The concentration of the FGF1 protein in the medium G is, for example, 10 ng to 1 μg/mL, preferably 10 ng to 500 ng/mL, more preferably 50 ng to 200 ng/mL, and further preferably about 100 ng/mL. When another FGF is used instead of FGF1 in procedure G, the concentration providing the same effect, with reference to FGF1, can be set.

When the WD progenitor-like cells used in procedure (E) are induced from human pluripotent stem cells, the medium G may further contain a BMP signal pathway inhibitor. Examples of the BMP signal pathway inhibitor that can be used in procedure G include the substances listed in procedure C, preferably LDN193189 or Noggin, and more preferably LDN193189.

When the medium G contains LDN193189, the concentration of LDN193189 in the medium G is preferably 1 nM to 300 nM, more preferably 1 nM to 100 nM, further preferably 5 nM to 20 nM, and still further preferably about 10 nM. When another substance is used instead of LDN193189 in procedure F, the concentration providing the same effect, with reference to LDN193189, can be set.

The culturing time in procedure G is not particularly limited, and is, for example, about 0.5 to 5 days, more preferably about 1 to 3 days. More preferably, when the WD progenitor-like cells used in procedure (E) are induced from mouse pluripotent stem cells, it is about 1 day, and when the WD progenitor-like cells used in procedure (E) are induced from human pluripotent stem cells, it is about 2 days.

In preferable embodiments, procedure G is a procedure of culturing cells obtained by procedure (F) (preferably cell aggregate) in a medium containing retinoic acid (preferably ATRA, and 10 nM to 1 μM, preferably 10 nM to 500 nM, more preferably 50 nM to 200 nM, and further preferably about 100 nM of ATRA), a Wnt agonist (preferably CHIR99021, and 0.1 μM to 300 μM, preferably 0.3 μM to 100 μM, more preferably 1 μM to 5 μM, and further preferably about 3 μM of CHIR99021), an ROCK inhibitor (preferably Y27632, and 1 μM to 1000 μM, preferably 1 μM to 100 μM, more preferably 1 μM to 50 μM, and further preferably about 10 μM of Y27632) and GDNF (0.1 ng to 100 ng/mL, preferably 0.2 ng to 20 ng/mL, more preferably 0.5 ng to 10 ng/mL, and further preferably about 2 ng/mL of GDNF) (preferably further containing a growth factor reduced Matrigel, and containing 5% to 50%, preferably 5% to 20%, more preferably 10% to 20%, and further preferably about 10% of a growth factor reduced Matrigel; and when the WD progenitor-like cells used in procedure (E) are induced from human pluripotent stem cells, further containing 10 ng to 1 μg/mL, preferably 10 ng to 500 ng/mL, more preferably 50 ng to 200 ng/mL, and further preferably about 100 ng/mL of FGF1; and when the WD progenitor-like cells used in procedure (E) are induced from human pluripotent stem cells, further containing a BMP signal pathway inhibitor (preferably LDN193189, and containing 1 nM to 300 nM, more preferably 1 nM to 100 nM, further preferably 5 nM to 20 nM, and more further preferably about 10 nM of LDN193189) (preferably suspension aggregate culture is performed, more preferably culturing as a suspension aggregate on a low adherent incubator; preferably culturing for about 1 to 5 days, more preferably culturing for about 1 day to 3 days).

In a preferred exemplary embodiment, the cells obtained in procedure G are PAX2, Emx2 and Ret positive and Hnf1b, Wnt9b and Calb1 positive.

### Exemplary Production of Kidney Organoid

The exemplary embodiments of the present disclosure can also provide a method for producing a kidney organoid, which comprises co-culturing ureteric bud-like cells produced using the method according to the exemplary embodiment(s) of the present disclosure with nephron progenitor cells and a stromal progenitor cell population. As the nephron progenitor cell, either embryonic nephron progenitor cells isolated from embryos or nephron progenitor cells induced from pluripotent stem cells (for example, ES cells or iPS cells) can be used. The method for inducing nephron progenitor cells from pluripotent stem cells can be referred to, for example, the report by the present inventors (A. Taguchi et al., Cell Stem Cell 14, 53-67, 2014). As the stromal progenitor cell population, for example, a stromal progenitor cell population isolated from an embryo can be used, and a stromal cell population sorted from an embryonic kidney can be used, though the means is not limited this. Preferably, a Pdgfra+ stromal cell population can be mentioned.

### EXAMPLES

Hereinafter, the exemplary embodiments of the present disclosure are described herein with reference to examples, but the present disclosure is not limited to the following examples.

The following animal experiments were conducted using a protocol approved by the Animal Care and Use Committee of Kumamoto University School of Medicine.

### Exemplary Materials and Methods

### Mouse

The Hoxb7-GFP mouse line was kept on an outbred background (Jcl: ICR, CLEA Japan, Inc.). Mice were maintained in a plastic cage with a 12-hour light cycle and fed a CE-2 irradiated diet. All analyses were performed with a minimum of 3 littermates. For the embryonic stage analyses, noon on the day when vaginal plugs of mated females were identified was regarded as embryonic stage (E) day 0.5. Hoxb7-GFP, which expresses GFP under the control of a fragment of the Hoxb7 promoter, was purchased from Jackson laboratory.

### Mouse ES Cell Culture Media

Mouse ES cell line (Osr1-GFP) (Taguchi et al., Cell stem cell 14, 53-67, 2014) was maintained on mitotically-inactivated murine embryonic fibroblasts (MEF) in DMEM supplemented with 15% FBS, 1% (v/v) non-essential amino acid (NEAA), 0.1 mM 2-mercaptoethanol (2-ME) and 1,000 U/mL leukemia inhibitory factor (LIF: Millipore). Prior to the initiation of differentiation, the ES cells were passages once onto a feeder cell-free gelatin-coated culture dish in DMEM supplemented with 15% FBS, 1% (v/v) non-essential amino acids, 0.1 mM 2-ME, 1×penicillin/streptomycin (P/S), 1,000 U/mL LIF, 3 μM CHIR99021 (Axon) and 1 μM PD0325901 (Wako).

Established mouse ES cell line (Hoxb7-GFP) was maintained on mitotically-inactivated MEF in GMEM supplemented with 14% KSR, 1% FBS, 1% (v/v) NEAA, 1% (v/v) sodium pyruvate, 0.1 mM 2-ME, 1,000 U/mL LIF, 1.5 μM CHIR99021 and 0.5 μM PD0325901. All mouse ES cell lines were cultured at 37° C. under a humidified atmosphere of 5% CO₂. Cells were passaged every other day.

### Mouse ES Cell Differentiation Media

The medium used was a mixture of 75% Iscove's modified Dulbecco's medium (IMDM) and 25% Ham's F12 medium, supplemented with 0.5×N2 (Thermo Fisher), 0.5×B27 without retinoic acid (Thermo Fisher), 0.5×P/S, 0.05% BSA, 2 mM L-glutamine, 0.5 mM ascorbic acid and 4.5×10-4 M 1-thioglycerol.

### Human iPS Cell Culture Media

Human iPS cells (201B7) were maintained on iMatrix-511 (Nippi, Inc.) in StemFit AKO3N media (Ajinomoto Co., Inc.). The human iPS cells were cultured at 37° C. under 5% CO₂ humidified atmosphere conditions. The cells were passaged every 6 days.

### Human iPS Cell Differentiation Media

A serum-free differentiation medium containing DMEM/F12 (Invitrogen) supplemented with 2% (v/v) B27 (without retinoic acid), 2 mM L-glutamine, 1% (v/v) ITS, 1% (v/v) NEAA (without retinoic acid), 90 μM 2-ME and 0.5×P/S was used.

### Establishment of Mouse ES Cell

Superovulated female 129/sv mice were mated with male Hoxb7-GFP mice to obtain fertilized 8-cell stage eggs. The harvested embryos were cultured in M2 medium (ARK Resource) for 24 hours. The embryos grown to the blastocyst stage were transferred to a 0.1% gelatin-coated plastic dish containing a mouse ES cell maintenance medium. Six days later, the propagated cells were passaged on mitotically-inactivated murine embryonic fibroblasts (MEFs) containing an ES cell maintenance medium. The established ES cells were further propagated, and the cells in the early passage were used for the differentiation experiments.

### Kidney Reconstitution Assay

The metanephroi were manually dissected from E11.5 ICR mouse embryos. The intact ureteric buds (UB) were isolated by incubating kidneys at 37° C. for 4 minutes in DMEM/10% FBS containing 1 mg/mL Type XI collagenase (SIGMA). The UBs were manually isolated from the metanephric mesenchyme (MM) using a 30 G needle. The harvested MM was washed in PBS once and dissociated by incubation with 0.05% trypsin/EDTA at 37° C. for 5 minutes. The dissociated MM cells were resuspended in a mouse ES cell differentiation medium to a concentration of 70,000 cells/100 μL, seeded into a low cell binding U-bottom plate (Thermo). The MM cells were precipitated by the centrifuge (1,000 rpm, 3 minutes). The isolated UB or Wolffian duct (WD), or induced UB, was placed onto the deposited sheet-like MM cells. The MM cells spontaneously aggregated and enrolled the UB, and finally formed spheroids after 24 hours cultivation. The reaggregated spheroids were transferred to a trans-well insert (Corning) containing 50% Matrigel in a DMEM/F12 medium (10% FBS and P/S) (50 μL), and then the transwell was inserted into a DMEM/F12 medium (10% FBS and penicillin/streptomycin).

### Culture of Stored Embryonic Cell

The posterior regions were harvested from the forelimbs of 22-26 somatic-stage embryos for embryonic tissue culture at F9.5. The harvested tissues were dissociated into single cells by incubating in DMEM/10% FBS containing 1 mg/mL Type XI collagenase for 6 minutes at 37° C., followed by treatment with DNase I and 0.25% trypsin at 37° C. for 6 minutes. After blocking in normal mouse serum, cell surface marker (Flk1) staining was carried out. Hoxb7-GFP+/Flk1− cells sorted by FACS were resuspended in a mouse ES cell differentiation media, and seeded in a V-bottom 96-well low cell binding plate (Sumitomo Bakelite, Co., Ltd., Cat #MS-9096V) so that there were about 1,600 cells per well. After the centrifuge (210 G, 4 min), the supernatant media was replaced with a medium containing 10 μM Y27632 (Wako), 0.1 μM retinoic acid, 3 μM CHIR99021, 5 ng/mL human Fgf9 (R & D), 1 ng/mL human GDNF (R & D) and 10% growth factor reduced Matrigel (BD) (“Procedure 6 medium”). After 24 hours, the aggregated spheroids were transferred to a medium containing 10 μM Y27632, 0.1 μM retinoic acid, 3 μM CHIR99021, 2 ng/mL human GDNF and 10% growth factor reduced Matrigel (“Procedure 7 medium”).

For embryonic tissue cultures at E8.75, the region caudal to the heart primordia of 12-15 somite-stage embryos were harvested and Hoxb7-GFP+/Flk1− cells were sorted by FACS. The sorted cells were aggregated in a V-bottom 96-well low cell binding plate at about 1,200 cells per aggregate. It was cultured for the first 24 hours using a medium containing 10 μM Y27632, 0.1 μM retinoic acid, 1 μM CHIR99021, 5 ng/mL human Fgf9 and 10% growth factor reduced Matrigel (“Procedure 5 medium”), then, the spheroids were transferred to “Procedure 6 medium” and “Procedure 7 medium,” and cultured for 24 hours each for differentiation.

Exemplary UB Lineage Induction from Mouse ES Cell

ES cells were differentiated in a serum-free medium as follows. The ES cells were dissociated with Accutase™ (ESGRO) and cultured in a serum-free mouse ES cell differentiation medium. The harvested cells were aggregated in a 96-well U-bottom low cell binding plate at 1,000 cells per aggregate to form embryonic bodies (EBs). After 48 hours (day 2), the EBs were dissociated with Accutase and re-aggregated in a serum-free differentiation medium supplemented with 10 ng/mL human activin A (R & D) (Procedure 1). After 24 hours (day 3), the medium was replaced with a medium containing 0.3 ng/mL human Bmp4 (R & D) and 10 μM CHIR 99021 (Procedure 2). After 36 hours (day 4.5), the medium was replaced with procedure 3 medium containing 0.1 μM retinoic acid, 100 ng/mL human Fgf9 and 10 μM SB431542 (Wako). On day 5.5 (24 hours later), the medium was replaced with a medium containing 0.1 μM retinoic acid, 100 ng/mL human Fgf9 and 5 μM CHIR99021 (“Procedure 4 medium”). The differentiation factors of the Hoxb7-GFP ES cell line are shown in the table below together with the differentiation factors of the Osr1-GFP ES cell line.

TABLE 1

| Nephron | DO-2 | D2-3 | D3-4.5 | D4.5-5.5 | D5.5-6.5 | D6.5-9.25 | | |
|---|---|---|---|---|---|---|---|---|
| Progenitor | Epiblast | Epiblast | Mesoderm | Mesoderm | Posterior IM | Metanephric NP | | |
| (mouse ESC) | induction | patterning | induction | maintenance | induction | induction | | |
| Osr-GFP | No factor | A1B0 | B0C10 | YB0C10 | YRA10B3C3 | YF5C1 | | |
| Hoxb7-GFP | No factor | A1B0 | B0C10 | YB0C10 | YRA10B3C3 | YF5C1 | | |
| Ureteric | D0-2 | D2-3 | D3-4.5 | D4.5-5.5 | D5.5-6.25 | D6.25-9.25 | | |
| Bud | Epiblast | Epiblast | Mesoderm | Anterior IM | Committed | WD maturation | | |
| (mouse ESC) | induction | patterning | Induction | Induction | WD Induction | 24 hr | 24 hr | 24 hr |
| Osr1-GFP | No factor | A10B0 | B03C10 | RF100SB10 | RC5F100 | YR | YR | YR |
| | | | | | | C1F5 | C3F5 | C3 |
| | | | | | | | G1 | G2 |
| Hoxb7-GFP | No factor | A3B0.3 | B0C10 | RC2F200 | RC3F100 | YR | YR | YR |
| | | | | | | C1F5 | C3F5 | C3 |
| | | | | | | | G1 | G2 |

Maturation Culture of Wolffian Duct (WD) Progenitor Cell Derived from Mouse ES Cell

On day 6.25, the induced spheroids were harvested and dissociated by incubating in 0.25% trypsin/EDTA at 37° C. for 6 minutes. After blocking in normal mouse serum, cell surface marker (CXCR4/KIT) staining was carried out in a buffer containing 1% BSA, 1×HBSS and 0.035% NaHCO₃. The FACS-sorted 3,000 Hoxb7-GFP+/CXCR4+/KIT+ cells were aggregated in a V-bottom 96-well low cell binding plate to form spheroids. Subsequently, these were cultured using the above E8.75 embryonic tissue culture conditions.

Branching Culture of Single Ureteric Bud Derived from Mouse ES Cell

On day 9.25, mouse ES cell-derived induced ureteric buds (UB) were manually isolated with a sharpened tungsten needle. The isolated UBs were embedded in 150 μL of a branching medium in a 24-well trans-well insert. The branch medium was a DMEM/F12 medium (Life Technologies) containing 50% Matrigel, 10% FBS, 0.1 μM retinoic acid, 100 ng/mL human Rspondin1 (R & D), 2 ng/mL human GDNF and 100 ng/mL mouse Fgf1 (R & D). The trans-well inserts were cultured in 500 μL of the branching medium without Matrigel.

Induction of MM Lineage from Mouse ES Cell

The previously reported MM lineage induction protocol for mouse ES cells (Taguchi, 2014 above) was applied with minimal modifications. Embryonic bodies (EBs) were formed by the 1,000 cells of aggregates in a 96-well U-bottom low cell binding plate. After 48 hours (day 2), the embryonic bodies (EBs) were dissociated with Accutase™ and then reaggregated in a serum-free differentiation medium supplemented with 1 ng/mL human activin A (R & D). After 24 hours (day 3), the medium was replaced with a medium containing 10 μM CHIR. After 36 hours (day 4.5), the medium was replaced with a fresh medium containing 10 μM Y27632 and 10 μM CHIR. On day 5.5, the medium was replaced with a medium containing 10 ng/mL activin A, 3 ng/mL Bmp4, 3 μM CHIR, 0.1 μM retinoic acid and 10 μM Y27632. On day 6.5, the medium was replaced with a medium containing 1 μM CHIR, 5 ng/mL human Fgf9 and 10 μM Y27632.

Induction of Ureteric Bud (UB) Lineage from Human iPS Cell

Cells were reaggregated at 10,000 cells per aggregate in a V-bottom 96-well low cell binding plate using a medium containing 10 μM Y27632 and 10 ng/mL human activin A and 1 ng/mL human Bmp4 (“Procedure 1 medium”), to form embryonic bodies (EBs). After 24 hours (day 1), the aggregates were transferred to a U-bottom 96-well low cell binding plate containing a medium containing 10 μM CHIR and 1 ng/mL human Bmp4 (“Procedure 2 medium”). After 36 hours (day 2.5), the medium was replaced with a medium containing 0.1 μM retinoic acid, 100 ng/mL human Fgf9, 100 nM LDN193189 and 100 μM SB431542 (“Procedure 3 medium”). On day 4.5, the medium was replaced with a medium containing 0.1 μM retinoic acid, 5 μM CHIR, 100 ng/mL human Fgf9 and 30 nM LDN193189 (“Procedure 4 medium”).

Maturation Culture of Wolffian Duct (WD) Progenitor Cell Derived from Human iPS Cell

On day 6.25, the induced spheroids were harvested and dissociated by incubating in 0.25% trypsin/EDTA for 6 minutes at 37° C. After blocking in normal mouse serum, cell surface marker (CXCR4/KIT) staining was carried out in a buffer containing 1% BSA, 1×HBSS and 0.035% NaHCO₃. The FACS-sorted 5,000 of CXCR4+/KIT+ cells were seeded in a V-bottom 96-well low cell binding plate and precipitated by the centrifuge (210 G for 4 minutes). The supernatant was replaced with a medium containing 10 μM Y27632, 0.1 μM retinoic acid, 1 μM CHIR, 5 ng/mL human Fgf9, 100 ng/mL human Fgf1, 10 nM LDN193189 and 10% growth factor reduced Matrigel (“Procedure 5 medium”). On day 8.5, the spheroids were transferred to a medium containing 10 μM Y27632, 0.1 μM retinoic acid, 3 μM CHIR, 5 ng/mL human Fgf9, 1 ng/mL human GDNF, 100 ng/mL human Fgf1, 10 nM LDN193189 and 10% growth factor reduced Matrigel (“Procedure 6 medium”). On day 10.5, the spheroids were transferred to a medium containing 10 μM Y27632, 0.1 μM retinoic acid, 3 μM CHIR, 2 ng/mL human GDNF, 100 ng/mL human Fgf1, 10 nM LDN193189 and 10% growth factor reduce Matrigel (“Procedure 7 medium”).

Branching Culture of Ureteric Bud Derived from Human iPS Cell

On day 12.5, induced ureteric bud spheroids derived from human iPS cells were embedded in 150 μL of branching medium in a 24-well trans-well insert. The branching medium was a DMEM/F12 medium containing 50% Matrigel, 10% FBS, 0.1 μM retinoic acid, 100 ng/mL human Rspondin1 (R & D), 2 ng/mL human GDNF, 100 ng/mL human Fgf1, 30 ng/mL human Fgf7 and 10 nM LDN193189. The trans-well inserts were cultured in 500 μL of branching medium without Matrigel.

Induction of MM Lineage from Human iPSC

The previously reported protocol (Taguchi 2014 above) was modified and applied. In the presence of 10 μM Y27632 and 1 ng/mL human activin A, cells were reaggregated at 10,000 cells per aggregate in a V-bottom 96-well low cell binding plate, to form embryonic bodies (EBs). After 24 hours (day 1), the aggregates were transferred to a U-bottom 96-well low cell binding plate containing the mesoderm-inducing medium containing 10 μM CHIR. Subsequently, half of the culture medium was replaced with a fresh medium every other day (day 3 and day 5). On day 7, the medium was replaced with ABC3R medium containing 10 ng/mL human activin A, 3 ng/mL human Bmp4, 3 μM CHIR and 0.1 μM retinoic acid. On day 9, the medium was replaced with ClF medium containing 1 μM CHIR and 5 ng/mL human Fgf9.

### Whole Mount Immunohistochemistry

Organoids were fixed in PBS containing 4% PFA for 60 minutes, washed with PBS containing 0.1% Triton X-100 for 3 times and blocked with PBS containing 10% goat serum, 1% Triton X-100 and 2% skim milk for 1 hour twice. The tissues were incubated overnight with primary antibodies and then incubated with secondary antibodies conjugated with Alexa Fluor 488, 568, 594, 633 or 647. After immunostaining, the tissues were cleared (Klingberg et al., J. Am Soc Nephrol 28, 452-459, 2017). The specimens were dehydrated with ethanol and replaced with ethyl cinnamate. The 3D fluorescent images were captured on 2-photon microscopy (FV1000-MPE; Olympus) or confocal microscopy (TSCSP8; Leica) and reconstructed by the software (Imaris; Bitplane or LASX; Leica).

### Section Immunohistochemistry

The samples were fixed in PBS containing 4% PFA for 60 minutes, washed with PBS, dehydrated with PBS containing sucrose, then embedded in OCT compound (TissueTek), and cryosectioned at 10 μm thickness. For fluorescent immunohistochemical analysis, the sections were incubated with primary antibodies, then incubated with second antibodies conjugated with Alexa Fluor 488, 568, 594, 633 or 647. The nuclei were counter-stained with DAPI. The fluorescent images were captured on confocal microscopy (TSCSP8; Leica).

### RNA Extraction, Reverse Transcription and Quantitative RT-PCR

Harvested spheroids or cells were homogenized, and total RNA was isolated using RNeasy Plus Micro Kit (Qiagen), and then a reverse-transcribed with random primers and Superscript III (Invitrogen). Quantitative PCR was carried out using Real-Time PCR system (Takara Bio) and Thunderbird SYBR qPCR Mix (Toyobo). With normalized by β-actin gene, relative mRNA expression levels were analyzed.

### Flow Cytometric Analysis Using Immunostaining

Cell aggregates induced from embryonic tissue or mouse ES cells/human iPS cells were dissociated and blocked with normal mouse serum and then cell surface marker staining was carried out in a buffer containing 1% BSA, 1×HBSS and 0.035% NaHCO₃. Data analysis was performed with FlowJo software (Treestar).

### Microarray Analysis

Microarray analysis were performed using an Agilent SurePrint G3 mouse gene expression (8×60K) microarray. The data were normalized by GeneSpring GX software (Agilent).

### Quantification and Statistical Analysis

All data analyses were performed by 3 independent experiments unless otherwise stated. The values were presented as mean±SE. Student's t-tests were applied for the statistical analysis of two groups.

### Exemplary Results

### Example 1

It was confirmed as follows that a robust branching capacity can be acquired by maturing the development of Wolffian duct (WD).

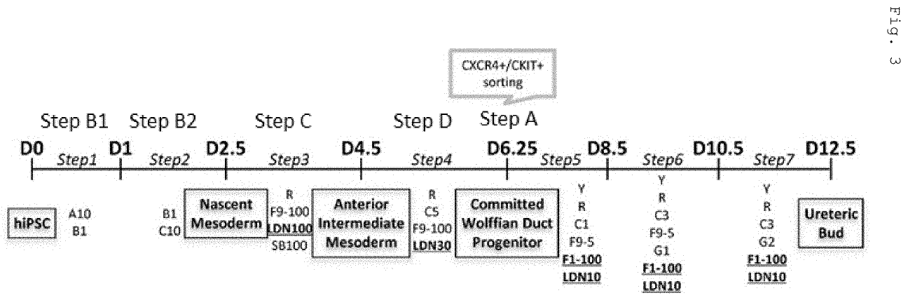

To evaluate the functional maturation process of early stage WD, we first employed the Hoxb7-GFP transgenic mouse line (Srinivas et al., Dev Genet 24, 241-151, 1999) to set up a kidney reconstruction assay system. FIG. 4 shows a schematic view of the WD development process. The portion of WD used for the reconstruction assay or microarray analysis is shown by the dashed line. At E8.75, it becomes a WD progenitors whose differentiation into UB lineage has been determined (“committed WD progenitor”). The isolated E11.5 metanephric mesenchyme (including nephron progenitor cells and stromal cells) were dissociated into the single cells and reaggregated together with WD or UB isolated from E9.5, E10.5 and E11.5 stage embryos. The number of branched tips of isolated UB or WD were counted at day 7 of the organ culture when each organoid stopped branching. The isolated UB or WD from E11.5 embryos showed the robust branch formation. On the other hand, the isolated WD from E10.5 and E9.5 stage embryos showed less branching numbers. The results are shown in FIG. 5. The final number of branches were not statistically different between WD at the caudal part and the rostral part of the E10.5 or E11.5 embryos. These results indicate that the branching capacity, which is retained regardless of the anterior-posterior position in the WD, is acquired by the development progression.

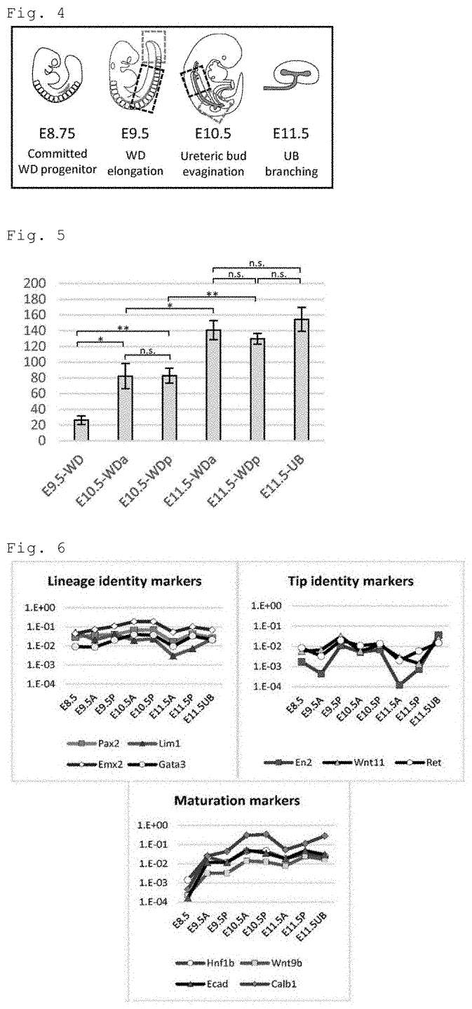

Gene expression array analysis was performed. Gene expression array analysis at each stage of UB, WD and their progenitor cells from E8.75 to E11.5 was performed to identify markers that could monitor the developmental maturation process. The results are shown in FIG. 6. Since the Hoxb7-GFP transgenic line leaks GFP fluorescence into the part of the vascular endothelial population, WD progenitors of Hoxb7-GFP+/Flk1-fraction were sorted by flow cytometry. Non-biased clustering analysis and similar entity analysis of representative UB marker genes identified several groups showing different gene expression kinetics. Many of the key transcription factors involved in early WD development (Pax2, Lhx1, Emx2, Siml, Gata3) were already expressed in the E8.75 committed WD progenitors and were maintained regardless of the developmental stages or anterior-posterior positions. Another group includes ureteral tip marker genes (e.g., En2, Wnt11, Ret), which showed higher expression at the leading tip legion of WD or UB. Conversely, a set of genes (e.g., E-cadherin, Wnt9b, Hnf1b) expression increased by the progression of development, which may be useful for monitoring the maturation.

### Example 2

It was then confirmed that retinoic acid, Wnt and Fgf/Gdnf signaling matures WD progenitors into ureteric bud-like cells as follows.

An exemplary protocol was established for generating UB by a reverse-induction approach. As the first procedure, the factors for maturing E9.5WD into E11.5UB-like cells were found as follows. The microarray analysis identified accumulated expression of retinoic acid synthetic enzyme (Raldh3), Wnt co-receptor (Lgr5) and Fgf receptor/target genes in the WD throughout the early development (data not shown). Hence, WD were sorted from the dissociated E9.5 mouse embryos and reaggregated in the presence of a combination of these growth factors. To support epithelial cell survival, Rho-kinase inhibitor (10 μM Y27632) and 10% growth factor reduced Matrigel were included.

The combination of RA, Wnt agonist (3 μM CHIR99021) and Fgf9 synergistically maintained the lineage markers (Pax2, Emx2) and tip type markers (Ret) and induced mature WD markers (Hnf1b, Wnt9b and Calb1) (FIG. 7). However, the aggregate (sphere) did not maintain the expression of Wnt11 and did not form buds morphologically. Therefore, it was attempted to employ Gdnf, a well-known upstream inducer of Wnt11, which is a strong inducer of ureteric buds. As expected, the conditions optimized employing Gdnf successfully induced the Wnt11 expression and bud-like structure formation (FIG. 5). These results are consistent with previously reported genetic loss-of-function studies showing the requirement of each growth factor signals, including Fgf, canonical Wnt signal and retinoic acid signal, during the WD development.

As the second procedure, the factors that mature the E8.75WD into the E9.5WD were found as follows. We investigated the factors that trigger maturation of the WD progenitors in the early stage, from embryonic E8.75 to E9.5 stage. At embryonic E8.75 to E9.5 stage, the Hoxb7-GFP positive WD progenitors are first clearly detectable in the anterior body trunk of the embryo (8-10 somite level). Similar to the induction from E9.5 to E11.5 stage, the sorted Hoxb7-GFP positive progenitor cells maintained the expression of Emx2 and Ret in the presence of retinoic acid (RA), Wnt agonist and Fgf9. In contrast, the same concentration of Wnt agonist and Fgf9 (3 μM and 100 ng/mL, respectively) worked inhibitory to the expression of maturation marker genes (Hnf1b, Wnt9b, Calb1, E-cadherin) at this procedure (FIG. 7). Therefore, it was concluded that the combination of RA and low concentrations of Fgf9 (5 ng/mL) and Wnt agonist (1 μM) was optimal for induction of this procedure. At this procedure, Fgf9 was able to maintain the expression of Wnt11 without using Gdnf. These results suggest that there is somewhat different mode of gene regulatory circuit between the early and late developmental stages of the WD maturation process.

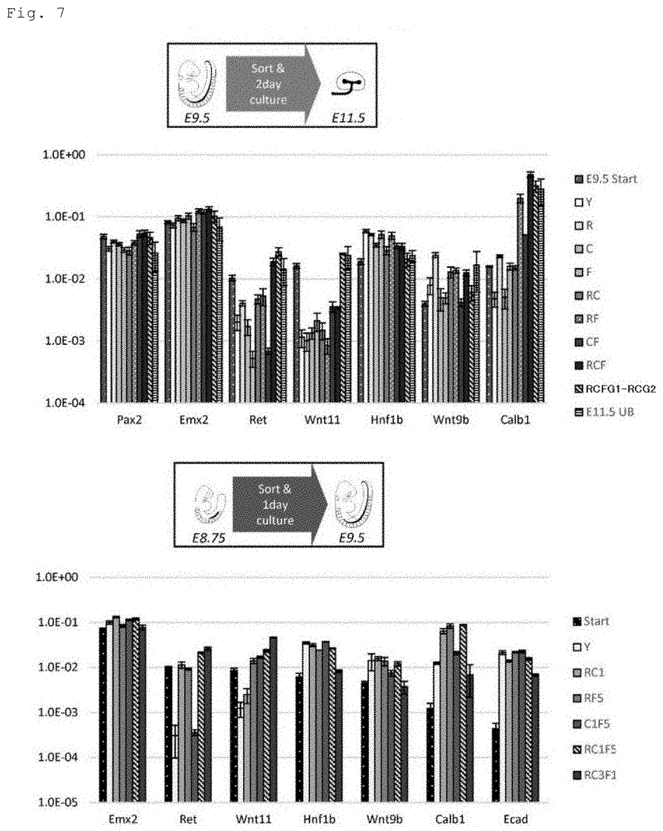

Finally, a 3-day induction protocol for maturing the E8.75WD into the E11.5UB-like cells, combining the above factors, was applied sequentially to the sored E8.75WD progenitors. The outline is shown in FIG. 1. At day 3 of induction, the formation of ureteric bud-like structure (FIG. 8) and the gene expression level quantitatively comparable to the embryonic UB (FIG. 9) were observed at the E11.5 embryos. The quantitative RT-PCR analysis identified the gene expression kinetics similar to those in vivo during the ex vivo maturing culture.

### Example 3

Using the E8.75WD progenitor-like population from mouse embryonic stem cells, the inducers of WD progenitors were searched for as follows.

First, in order to specifically and quantitatively evaluate the efficiency of WD progenitor induction, we searched for a combination of cell surface molecules specifically expressed in the E8.75WD progenitors. Comparing the genes expressed in Wolff progenitor cells and ureteric bud cells (E8.75WD, E9.5WD_C, E10.5WD_R, E10.5WD_C, E11.5UB) and metanephric mesenchymal lineage progenitor cells (E9.5IM_R, E9.5IM_C) at each stage of development, we focused on CXCR4, Icam2, Itgb3 and KIT as molecules expressed from the earliest Wolffian duct of E8.75, which are not expressed in the metanephric mesenchymal lineage.

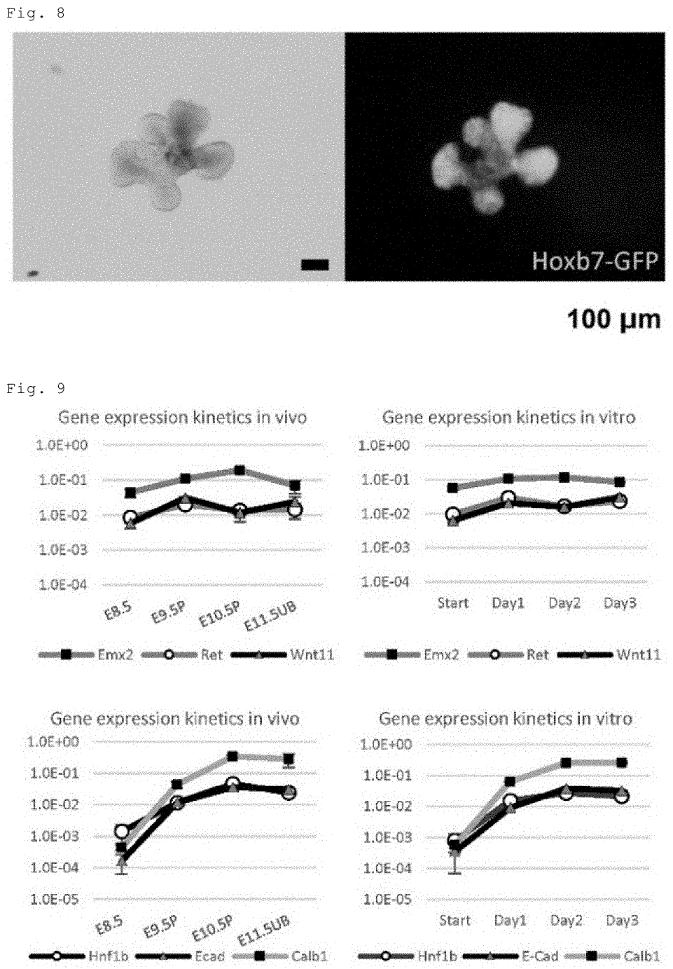

E8.75 fetuses of mice expressing GFP in Wolffian duct were dissociated into single cells, and surface molecules expected to be expressed in WD were analyzed by flow cytometry. FACS analysis using Itgb3 showed that most of the Hoxb7-GFP+ metanephric mesenchymal lineage progenitor cells were Itgb3 negative. In addition, FACS analysis performed using Icam2 revealed that Icam2 is strongly expressed in the vascular endothelium, but only a small part is weakly expressed in WD. FACS analysis using CXCR4 and KIT revealed that KIT was slightly weakly expressed in the vascular endothelium, but CXCR4 was expressed only in WD, and 99% or more of CXCR4-positive/Hoxb7-GFP-positive WD were KIT-positive. The majority of the E8.75 Hoxb7-GFP-positive/Flk1-negative WD progenitors are positive for CXCR4 and KIT, and the population of WD progenitors in the whole cells contained in the E8.75 embryo and the CXCR4-strongly positive and KIT-strongly positive population was consistent, and specificity was identified as the CXCR4 and KIT markers (circles in FIG. 10).

### Example 4

The induction of UB lineage (AIM) from T-positive immature mesodermal state was investigated.

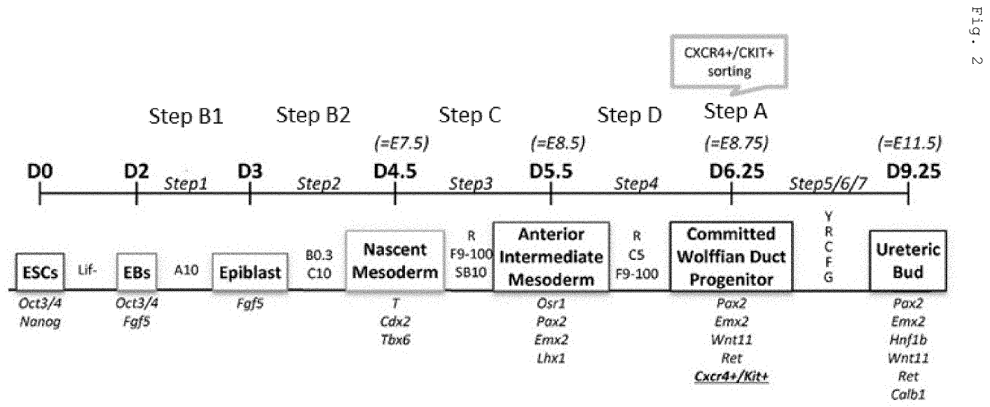

In order to induce WD progenitors from mouse embryonic stem cells, we first examined the in vivo A-P patterning process within the intermediate mesoderm. The lineage segregation model of UB and MM already constructed (see Taguchi, 2014 above) indicated that the UB lineage differentiates earlier than the MM lineage from the T-positive immature mesoderm state, and the immature state is maintained by strong Wnt signaling. Therefore, first, the incubation period with a high-concentration Wnt agonist was tentatively shortened to 1.5 days for UB induction, while 2.5 days for MM induction (procedure 2 in FIG. 2).

Next, signaling for the AIM differentiation procedure was hypothesized, which is partially different from that for PIM, based on the gene expression profile minimally conserved, between anterior intermediate mesoderm (AIM) (Osr1+, Pax2+, Pax8+, Emx2+, Lhx1+, Gata3+) and posterior intermediate mesoderm (PIM) (Osr1+, Wtl+, Hoxll+) (Procedure 3 in FIG. 2).

First, retinoic acid was mentioned as the common inducer for both AIM and PIM induction. The endogenous FGF signal was sufficient for PIM induction, but high concentrations of Fgf9 further enhanced AIM markers. In contrast to the PIM induction, addition of activin A and Bmp4 was inhibitory to the induction of AIM markers. Suppression of the smad 2/3 pathway by SB431542 (SB) enhanced the AIM marker induction. This suggests the principle role of activin/Tgfb signaling for AIM vs. PIM fate determination. On the other hand, either addition or inhibition of Bmp signal worked inhibitory for the AIM induction. This means that the AIM specification requires an optimal level of Bmp signal.

Next, the factors that specify the AIM to the WD progenitors with CXCR4+/KIT+ were investigated (FIG. 2: procedure 4). The results are shown in FIG. 11. At this procedure, it was found that the synergistic effect of RA, Wnt agonist and Fgf9. In particular, removal of the Wnt agonist dramatically reduced the induction of the CXCR4+/KIT+ population. This suggests that the Wnt agonist plays a crucial role in the induction of WD progenitors.

For further fine-tuning of the conditions and understanding the UB lineage differentiation process, we re-examined and changed the incubation period with Wnt, thereby identified the optimal timing for efficient differentiation of nascent mesoderm into AIM (FIG. 2: procedure 2). The permissive time window for the AIM induction was approximately 4.5 days (36 h of Wnt treatment). At the timing of day 4 (procedure 2: period is 1 day) or day 5 (procedure 2: period is 2 days), the efficiency decreased dramatically. This may reflect the antero-posteriorly very narrow anterior intermediate mesoderm domain (pronephric anlagen) which initially appears within the 2-somite width (somite level 8-10) in the intermediate mesoderm at E8.5 in vivo.

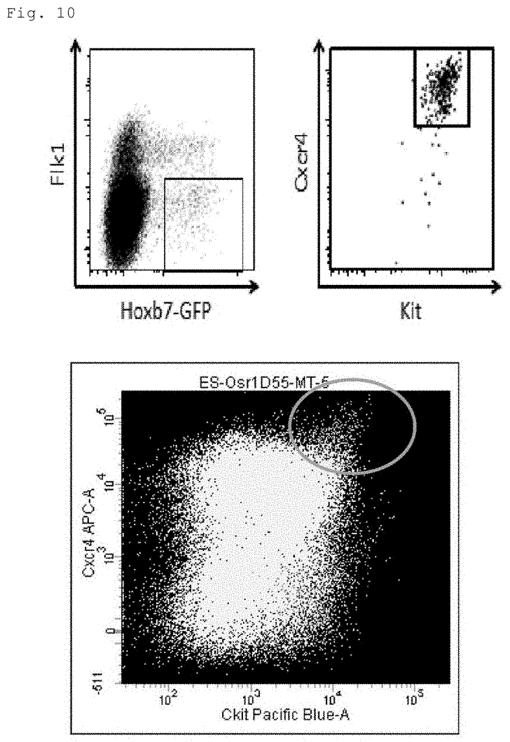

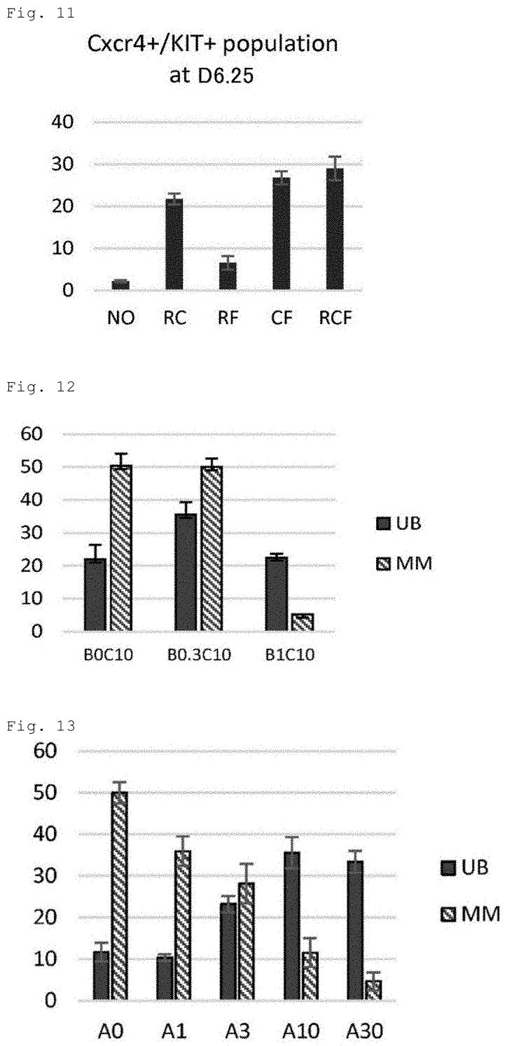

From day 2 to day 3 (Procedure 1) and day 3 to day 4.5 (Procedure 2) of differentiation, concentration-dependent patterning by the activin/Bmp signaling was observed, so, fate-specific signals in epiblast and primitive streak/early mesoderm stage were investigated. In mesoderm formation/patterning (Procedure 2), UB induction was maximized at higher Bmp4 concentrations compared to MM (FIG. 12). At the epiblast patterning stage (Procedure 1), the higher concentration of activin A was preferred for UB induction, as compared with MM (FIG. 13). The combination analysis of these two procedures showed the reciprocal pattern in the optimal concentration range for induction to UB and MM (FIG. 14). These results suggest that the cell fate patterning of UB and MM begins before and during the formation of immature mesoderm. These optimizations (optimization of procedure 1 and procedure 2) resulted in an average 35.6% of CXCR4+/KIT+ population at day 6.25 of differentiation of mouse ES cells.

The gene expression kinetics from the immature ES cells at day 0 to the WD progenitor stage at day 6.25 (corresponding to E8.75 WD progenitors) was analyzed. The results are shown in FIG. 15. The induced spheroids showed quantitatively comparable gene expression level to E8.75 WD progenitors and lacked expression of PIM or metanephric nephron progenitor cell markers (Hoxdll, Wtl and Six2). This indicates the successful selective induction to the UB lineage.

### Example 5

Using the induced UB, the superstructure of the embryonic kidney was reconstructed as follows.

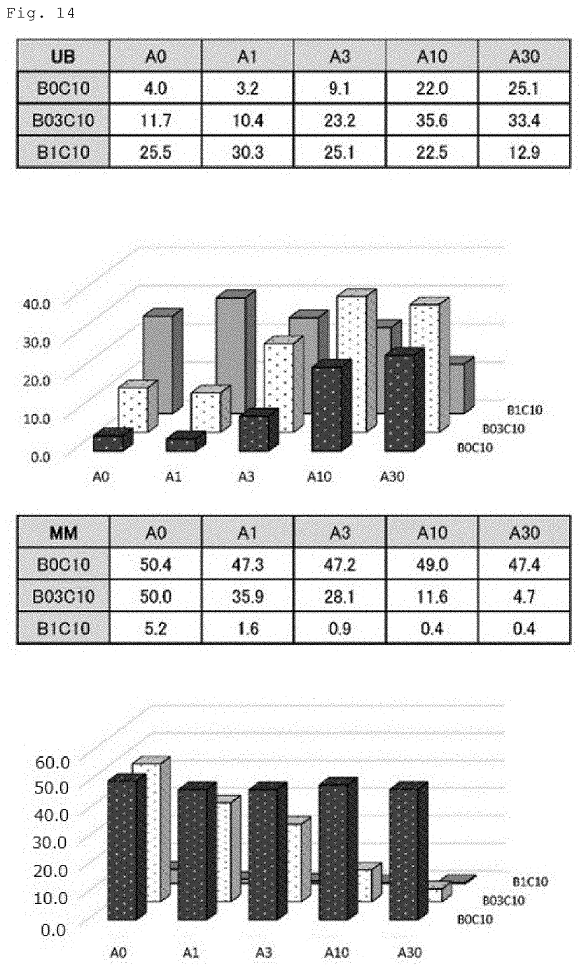

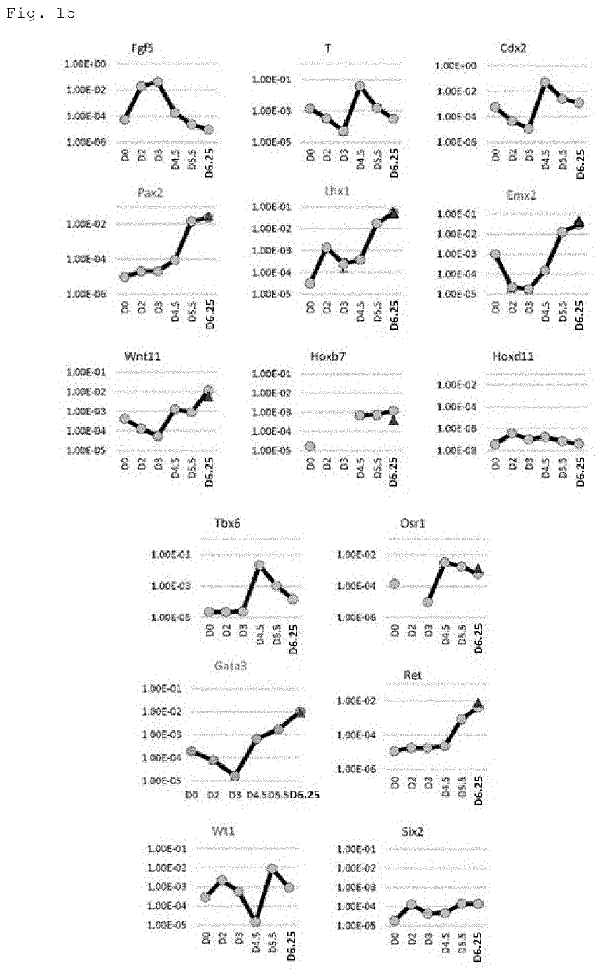

Next, the WD maturation factors for the induced WD progenitors were examined. Mouse embryonic stem cells were established from Hoxb7-GFP transgenic mice to visualize branching morphogenesis. Minimal modifications in the initial induction procedures successfully induced the Hoxb7-GFP+/CXCR4+/KIT+ WD progenitors at day 6.25 of differentiation. The sorted GFP+/CXCR4+/KIT+ cell population was reaggregated and cultured under the WD maturation condition established by the E8.75 WD culture experiments shown in FIG. 16. The aggregates formed ureteric bud-like structures at day 9.25 of induction (FIG. 17) and expressed UB markers which are comparable to that of E11.5 UB level (FIG. 18). Single straight or bifurcated bud was manually isolated from the spheroid and reaggregated with the isolated E11.5 MM to create a single exit and confirm the branching capacity. In the presence of MM, the induced UB underwent dichotomous branching up to 6th to 7th generation in 6 to 7 days of organ culture (1 generation/day) (FIG. 19, left figure). The final number of tips from the single bud grew 141±12 in average (n=6), which was comparable to that of E11.5 embryo-derived UB (FIG. 19, right figure). By modifying the method described in the previous report (Rosines et al., Hum Mol Genet 20, 1143-1153, 2007), the branching capacity of induced UB was assessed under cell-free branching culture conditions. As a result, the optimized medium contained RA, Fgf1, Wnt agonist (Rspo1), Gdnf and 10% FBS in the presence of 50% Matrigel.

To further verify the functionality of the induced UB, the capacity to maintain the progenitor cell niche and differentiation capacity were analyzed by the whole mount staining of the reconstituted organoid at day 7.

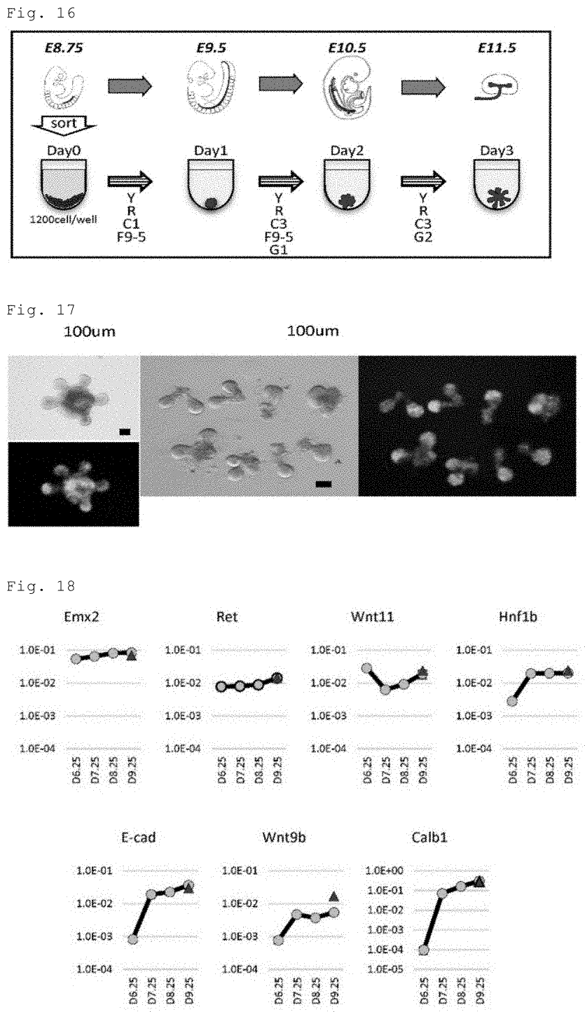

The induced UB maintains the Six2-positive nephron progenitor cells on each UB-tip in the periphery of the organoid, which corresponds to the nephrogenic zone of the embryonic kidney (FIG. 20, left figure). In contrast, the inner side of the organoid, differentiated nephrons were observed that sequentially contained E-Cadherin-positive distal tubule segment, LTL-positive proximal tubule segment, and Nephrin-positive glomerulus structure. From this, the nephron induction capacity of the induced UB was confirmed (FIG. 20, right figure). The distal end of each nephron was connected to the ureteric tip, which is essential to interconnect nephrons for the urine drainage (FIG. 20, bottom right figure).

Sox9, a typical UB tip marker, was expressed at the periphery (data not shown). On the other hand, cytokeratin 8 showed stronger expression in the medullary region of the kidney, similar to the embryonic kidney at E14.5. This indicates that the proper tip-stalk patterning takes place. The ubiquitous expression of Calb1 and Gata3 in the entire ureteric epithelium further confirmed the ureteric lineage specific feature of this ramified epithelium (data not shown).

In summary, the induced UB fulfills the functional criteria of the UB, including the branching morphogenic capacity, the maintenance capacity of nephron progenitor cells and the nephron differentiation capacity. This indicates that it has become enabling the reconstruction of the superstructure of the embryonic kidney with metanephric mesenchyme.

### Example 6

The use of induced nephron progenitor cells instead of embryonic MM was tested.

Since the inducing nephron progenitor cells inducible condition was already establish, it was tried to replace the embryonic MM with the induced nephron progenitor cells. First, the individual requirement of MM composing population; namely, the nephron progenitor cells and stromal progenitor cells, was tested. As already revealed, most of the nephron progenitor cells at E11.5 reside in the Itga8+/Pdgfra-fraction, whereas the stromal progenitor cells exhibit Pdgfra (Taguchi et al., Cell stem cell 14, 53-67, 2014). Therefore, each single fraction was sorted and reaggregated with the induced UB. The stromal cells did not support the UB branching in the absence of nephron progenitor cells. On the other hand, the nephron progenitor cells induced irregular branching formation without stromal cell populations and the tips of UB, which shows multi angular shape rather than typical bifurcation or trifurcation, were formed (data not shown). In addition, on each site of the UP trip, irregularly thickened layers of nephron progenitor cells were observed (data not shown). This is a reminiscent of the previously reported knockout mouse phenotype which lacks the functional stromal population. These facts indicate that both the stromal and nephron progenitor cell population are required for the proper organization of the ureteric bud branching morphogenesis. Therefore, we decided to reconstruct kidney organoid using induced nephron progenitor cells, induced UB and Pdgfra+ stromal cell population sorted from E11.5 embryonic kidneys. Similar to the reconstruction with whole embryonic MM, the reconstructed kidney tissue showed robust branching with nephron progenitor cell niche and the differentiated nephron components. The functionality of both of the mouse ES cell-derived nephron progenitor cells and ureteric buds has been demonstrated, indicating that they can interact to form superstructure of the kidney organoid (FIG. 21). CAG-delta-Tomato mouse embryos were used for the Pdgfra+ stromal cell population to confirm the selective contribution of each population. It showed the restricted contribution of the embryonic cells to the stromal population surrounding the nephron progenitor cell niche and medullary region of the organoid (data not shown).

### Example 7

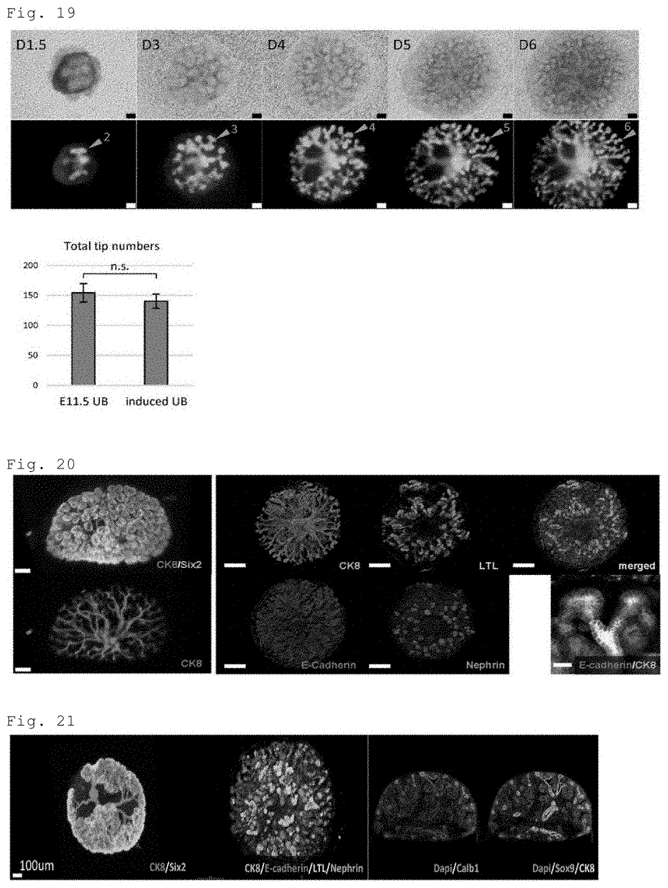

The induction of WD from human iPS cells was performed as follows. With reference to experiments using mouse ES cells, nascent mesoderm was first induced by activin A and following high concentration of Wnt agonist (FIG. 3, procedure 1 and procedure 2). Then, AIM induction factors and WD induction factors were composed in each procedure by combining RA, Fgf9 and Tgfb inhibitors or RA, Wnt agonist and Fgf9, respectively (Procedure 3 and procedure 4). In contrast to the induction of mouse ES cells, addition of the Bmp inhibitor LDN further enhanced in both of these differentiation procedures. In particular, the administration of LDN at AIM induction procedure (Procedure 3) greatly enhanced the AIM marker genes expression and induction of CXCR4+/KIT+ WD progenitors. This was confirmed by quantitative analysis at day 4.5 and day 6.25, respectively (FIG. 22).

Next, the optimal time window for inducing anterior or posterior intermediate mesoderm from the nascent mesoderm was examined. Since the nascent mesoderm population is maintained by the high concentrations of Wnt agonists, the incubation period with Wnt agonists was varied and the subsequent differentiation was applied with conditions specific to the UB or metanephric nephron progenitor cell lineage. The induction efficiency of CXCR4+/KIT+ WD progenitor or ITGA8+/PDGFRA− nephron progenitor cell fraction was examined at day 6.25 or day 12, respectively. Similar to the mouse ES cell experiments, the human UB lineage also required the rigid time window for the AIM induction, which was optimal at day 2.5 (CHIR treatment for 1.5 days). On the other hand, the induction efficiency of nephron progenitor cell lineage peaked by the day 7.5 administration (CHIR treatment for 6 days) of the PIM induction factor, confirming the previous report (see, e.g., Taguchi 2014 above). UB lineage was not induced beyond the optimal time window, compared to the permissible time window for the CHIR treatment for the induction of nephron progenitor cells (between 5 and 7 days). Therefore, the UB lineage was not induced in the time frame for differentiation of nephron progenitor cells.

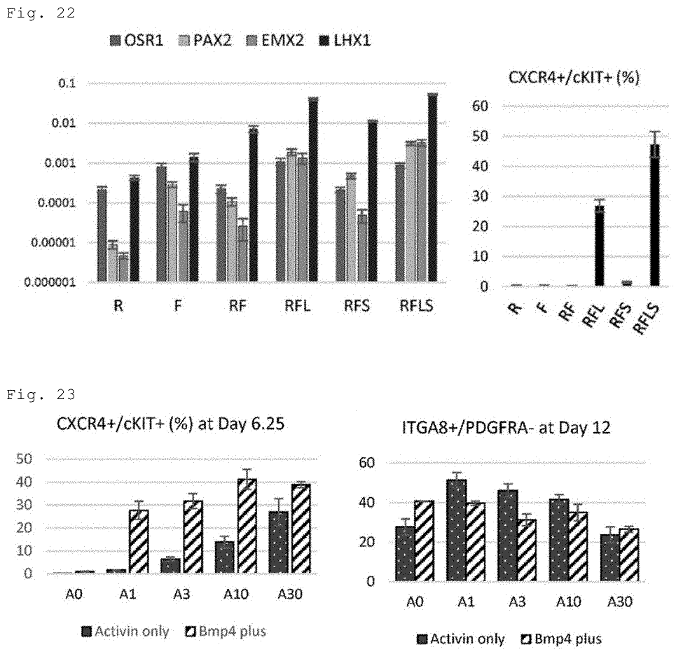

Since the mouse ES cell experiments indicated that premature patterning during the epiblasts to nascent mesoderm stage affects the fate determination of UB vs. nephron progenitor cells, the optimization of the activin/Bmp concentration at the earlies phase of the differentiation was investigated. At the epiblast patterning stage (day 0 to day 1 of differentiation), the UB preferred the higher activin signals, coincident with the mouse ES cell results, and further, by the addition of low concentrations of Bmp4 at this procedure, the WD progenitor induction was further enhanced and peaked with the combination of 10 ng/mL activin A and 1 ng/mL Bmp4 (FIG. 23, left figure, FIG. 24). In contrast, in the absence of Bmp4, lower concentrations of activin A enhanced the induction of nephron progenitor cells, which maximized with 1 ng/mL activin A (FIG. 23, right figure, FIG. 24).

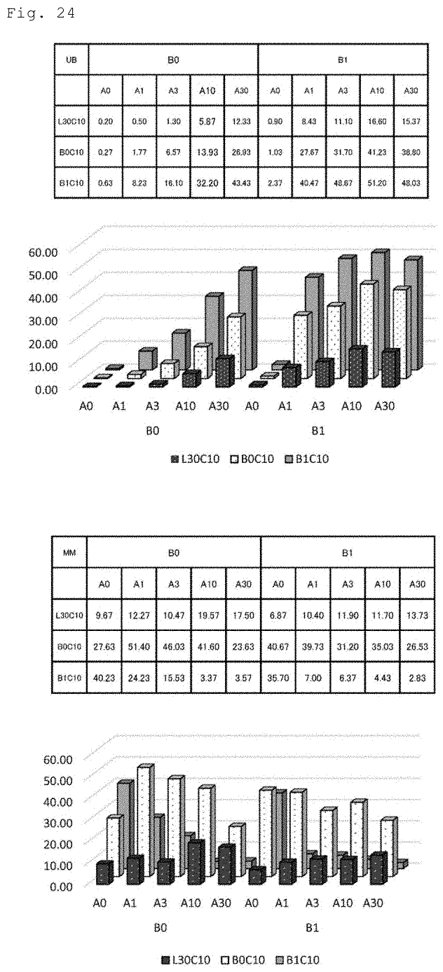

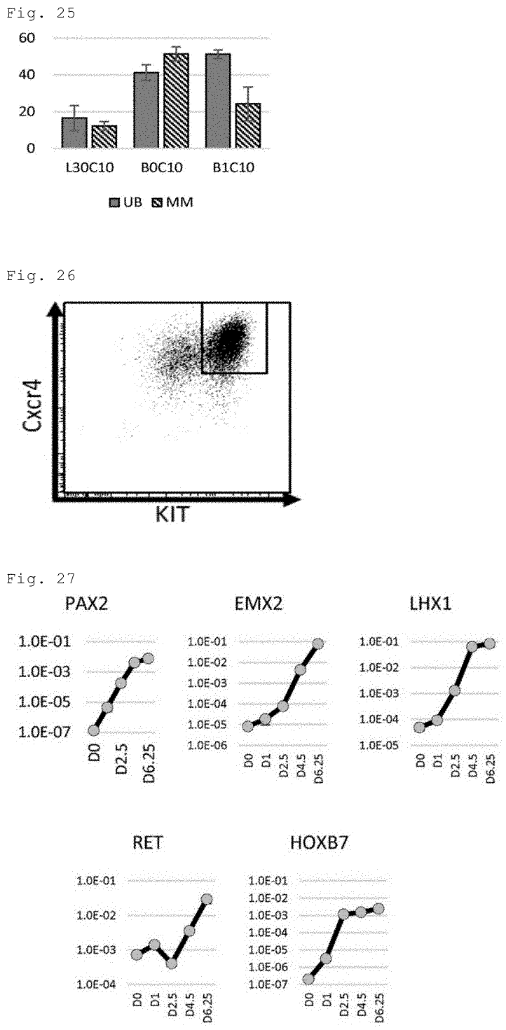

The effect of co-administrated Bmp signal during the subsequent mesoderm induction/patterning procedure by the CHIR treatment (days 1 to 2.5 for UB induction and days 1 to 7 for MM induction) was examined. The UB progenitor cells were most efficiently induced by the addition of 1 ng/mL Bmp4, while the nephron progenitor cells were most efficiently induced in the absence of Bmp ligand or antagonist (FIG. 25). Taken together, these results strongly suggest that there is the mutually independent early lineage specification process between UB and metanephric nephron progenitor cell lineage. Under the finalized condition, approximately 51.2% of the CXCR4+/KIT+ WD progenitors were induced at day 6.25 of the induction (FIG. 26), and the kinetic analysis from day 0 to day 6.25 showed that the set of WD markers was induced efficiently (FIG. 27).

Next, the CXCR4+/KIT+ WD progenitor fraction was sorted at day 6.25, and reaggregated in the presence of 10% Matrigel and WD maturation factors. For the differentiation of human WD progenitors, continuous administration of Fgf1 and LDN in addition to mouse WD maturation cocktails further enhanced the expression of the mature WD marker genes (data not shown). The optimized culture condition induced the mature UB markers including Hnf1b, E-Cadherin and CALB1 (data not shown), and formed the multiple bud formation at day 6 of cultivation (totally day 12 of induction) (FIG. 28).

The induced UB showed branching capacity in the gel culture environment. Under this culture condition, the first branching was observed approximately at day 7 of culture, and the second generation of bifurcation was detected at the end of the second week (FIG. 28). This is considerably slower compared to mouse UB branching, but compatible to the in vivo human development. The branched UB organoid expressed Sox9 in the tip region and CK8 in the stalk region of the ureteric epithelium (FIG. 29, left figure). This indicates the tip-stalk patterning in structure. In the tip region, the typical dichotomous bifurcation stained with the E-cadherin and PAX2 was identified (FIG. 29, right figure). This demonstrates the first evidence that the human UB branching morphogenesis was reconstructed in vitro.

### Example 8

Since the selective induction method of MM and UB developed by according to the exemplary embodiments of the present disclosure facilitates the lineage specific role of developmental genes, we attempted to examine the cell-autonomous role of PAX gene in the MM and UB lineage using this method.

Deletion of PAX2 from the MM lineage did not show the gross abnormality at least nephron progenitor cell induction as well as mesenchymal-to-epithelial transition toward the nephrons. In contrast, in the UB lineage, we observed the phenotypes in which UB differentiation was incomplete. At day 6.25 of differentiation, knockout clones showed a slight decrease in the induction efficiency of CXCR4+/KIT+ WD progenitors compared to controls (data not shown). Nevertheless, the results of the quantitative RT-PCR analysis showed genetically comparable WD marker gene expression profiles except for PAX2, and significant percentage of CXCR4+/KIT+WD progenitors were obtained both from the control and the PAX2 knockout clones (data not shown).

The sorted CXCR4+/KIT+ WD progenitor population was further cultured under the WD maturation condition. At day 8.5 (at day 2 of maturation culture), morphological macroscopic differences were not observed between the control and knockout clones. At day 10.5 (at day 4 of maturation culture), the control clones showed the active cellular protrusion formation, which are reminiscent of migrating WD tips in morphology (Soofi et al., 2012), while the knockout clones showed less protrusion and gradual decrease in the expression of PAX2 target genes, including LHX1, GATA3 and RET (data not shown).

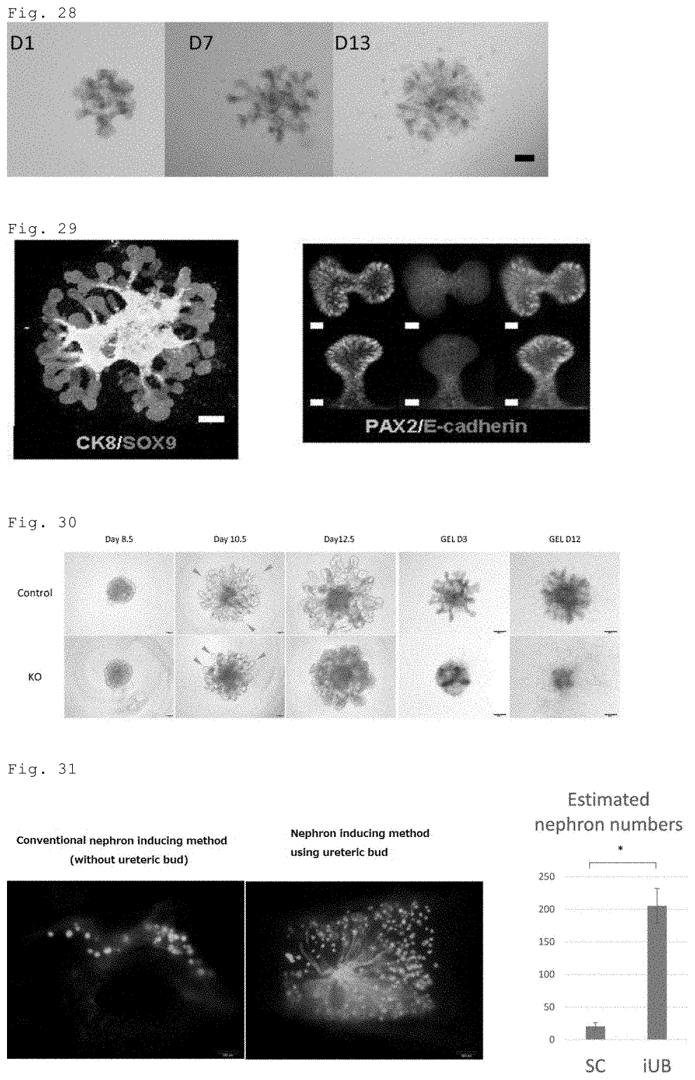

At day 12.5 (at day 6 of maturation culture), the control spheroid developed into the massive ureteric bud-like structure, while the knockout clones showed rough surface without clear bud formation (FIG. 30). The expression level of E-cadherin in the knockout clones was significantly lower compared to the control clones. It suggests the failure of proper mesenchymal-to-epithelial transition in the knockout clones. Analysis of day 12.5 spheroid by the whole mount immunostaining revealed the clear accumulated E-cadherin signaling in the extracellular membrane of the basal side region of the ureteric bud in the control. In contrast, the knockout clones showed the weak cytoplasmic expression of the E-cadherin without membrane localization.

This means that the decrease of PAX2 in the UB lineage results in a failure to induce the epithelial-to-mesenchymal transition during the maturation stage.

### Example 9

The influence on induction of differentiation from nephron progenitor cells to nephrons was compared with the case of using ureteric bud-like cells produced using the method according to the exemplary embodiment(s) of the present disclosure and the case of using the conventional method for co-culturing with feral spinal cord tissue (without ureteric bud).

Using the ureteric bud-like cells produced according to the method described in the examples, nephrons were induced as follows. Metanephric mesenchyme harvested from a fetal mouse in which glomerular epithelial cells emit fluorescence of GFP was aggregated with induced ureteric bud-like cells, and organ culture was conducted for 7 days, then, these were transplanted into immunodeficient mice. At day 15 of transplantation, the cells were harvested, and the number of GFP-positive glomeruli that were recognized as spherical structure was counted, and the total number of finally formed nephrons was estimated.

As control, organ culture was performed using the method in which the fetal spinal cord tissue and the metanephric mesenchyme were co-cultured described in the report by the present inventors (A. Taguchi et al., Cell Stem Cell 14, 53-67, 2014), and the resultant cells were transplanted.

The results are shown in FIG. 31. It was found that the number of nephrons finally formed was significantly increased by maintaining the nephron progenitor cells using the ureteric bud-like cells produced using the method according to the exemplary embodiment(s) of the present disclosure.

### Exemplary Industrial Applicability

According to the method according to the exemplary embodiment(s) of the present disclosure, it can become possible to produce ureteric bud-like cells in vitro, which is comparable to fetal ureteric buds. The functional features comprise, forming a kidney organoid having a dendritically branching capacity, maintain a progenitor cell niche at the tips of the branches, and connect individual nephrons by the branches.

The exemplary method for producing WD progenitor-like cells provided by the present disclosure enables production of ureteric bud-like cells having following features. (I) When mixed with metanephric mesenchyme or cultured in a growth factor which promotes branching, they undergoes branching. (II) When mixed with metanephric mesenchyme, they show an ability of forming a kidney progenitor niche, which maintains undifferentiated nephron progenitors. (III) When mixed with metanephric mesenchyme, they show an ability of differentiating nephron progenitor cells into the nephrons.

In the presence of nephron progenitors and stromal progenitors, the ureteric bud-like cells produced using the method provided by the present disclosure can form a kidney organoid, which contains differentiated nephrons connected by dendritically branching collecting duct structures. The kidney organoid presented described in the exemplary embodiments herein shows the first evidence of reproducing the higher-order structure of the kidney in the world. Hence, the exemplary embodiments of the present disclosure can be an indispensable technique for facilitating a production of a functional artificial kidney.

1-16. (canceled)

17. A method for producing Wolffian duct (WD) progenitor cell-like cells capable of being differentiation-induced into ureteric bud-like cells, comprising:
(a) culturing first pluripotent stem cells as second pluripotent stem cells in a first medium comprising activin A or a tumor growth factor;
(b) culturing the second pluripotent stem cells as third pluripotent stem cells in a second medium comprising a Wnt agonist;
(c) culturing the third pluripotent stem cells as fourth cells in a third medium comprising (i) a retinoic acid or a retinoic acid analog, (ii) a fibroblast cell growth factor, and (iii) a TGFβ signal pathway inhibitor or a Wnt agonist,
(d) culturing the fourth cells as fifth cells in a medium comprising (i) a retinoic acid or a retinoic acid analog, (ii) a Wnt agonist, and (iii) a fibroblast cell growth factor; and
(e) obtaining C-X-C chemokine receptor 4 (Cxcr4)-positive and KIT proto-oncogene receptor tyrosine kinase (KIT)-positive cells from the fifth cells,
wherein the components in each of procedures (a)-(d) can be the same substance or different substances.

18. The method according to claim 17, wherein procedure (d) is a cell sorting procedure in which the proportion of the Cxcr4-positive and KIT-positive cells is 30% or more in all cells.

19. The method according to claim 17, wherein the Cxcr4-positive and KIT-positive cells express at least two genes selected from the group consisting of Paired box (Pax) 2, LIM homeobox (Lhx) 1, empty spiracles homeobox (Emx) 2, ret proto-oncogene (RET) and homeobox (HOX) B7.

20. The method according to claim 17, wherein procedure (d) is performed by sorting Cxcr4-positive and KIT-positive cells.

21. The method according to claim 17, wherein at least one of the first, second and third pluripotent stem cells is a human iPS cell.

22. The method according to claim 17, wherein the first medium comprises 1 to 1000 ng/mL of activin A and the second medium comprises 1 to 1000 μM of CHIR99021.

23. The method according to claim 22, wherein a time for performing procedure (b) is 1 day to 2 days.

24. The method according to claim 17, wherein the second medium further comprises a BMP signal pathway active substance.

25. A method for producing ureteric bud-like cells, comprising:
(a) culturing first cells which are first C-X-C chemokine receptor 4 (Cxcr4)-positive and KIT proto-oncogene receptor tyrosine kinase (KIT)-positive WD progenitor cell-like cells to provide second cells in a first medium containing (i) retinoic acid or a retinoic acid analog, (ii) a Wnt agonist, (iii) FGF9, and (iv) a ROCK inhibitor;
(b) culturing the second cells to provide third cells in a second medium comprising (i) retinoic acid or a retinoic acid analog, (ii) a Wnt agonist, (iii) a fibroblast cell growth factor, (iv) a ROCK inhibitor, and (v) a glial cell line-derived neurotrophic factor (GDNF) or a GDNF analog, and
(c) culturing the third cells obtained in a third medium comprising (i) retinoic acid or a retinoic acid analog, (ii) a Wnt agonist, (iii) a ROCK inhibitor, and (iv) GDNF or a GDNF analog.

26. The method according to claim 25, wherein the ureteric bud-like cell express Hnf1b, E-Cadherin and CALB1.

27. The method according to claim 25, wherein the C-X-C chemokine receptor 4 (Cxcr4)-positive and KIT proto-oncogene receptor tyrosine kinase (KIT)-positive WD progenitor cell-like cells are obtained by:
(i) culturing first pluripotent stem cells as second pluripotent stem cells in a fourth medium comprising activin A or a tumor growth factor;
(ii) culturing the second pluripotent stem cells as third pluripotent stem cells in a second medium comprising a Wnt agonist, optionally further comprising a BMP signal pathway active substance;
(iii) culturing the third pluripotent stem cells as fourth cells in a third medium comprising (i) a retinoic acid or a retinoic acid analog, (ii) a fibroblast cell growth factor, and (iii) a TGFβ signal pathway inhibitor or a Wnt agonist,
(iv) culturing the fourth cells as fifth cells in a medium comprising (i) a retinoic acid or a retinoic acid analog, (ii) a Wnt agonist, and (iii) a fibroblast cell growth factor; and
(v) obtaining the C-X-C chemokine receptor 4 (Cxcr4)-positive and KIT proto-oncogene receptor tyrosine kinase (KIT)-positive cells from the fifth cells,
wherein the components in each of procedures (i)-(iv) can be the same substance or different substances.

28. A method for producing a kidney organoid, comprising co-culturing ureteric bud-like cells induced from C-X-C chemokine receptor 4 (Cxcr4)-positive and KIT proto-oncogene receptor tyrosine kinase (KIT)-positive WD progenitor cell-like cells with nephron progenitor cells, and embryonic kidney-derived Platelet Derived Growth Factor Receptor Alpha (Pdgfra)-positive stromal cells.

29. A method according to claim 28, wherein the ureteric bud-like cells are obtained by:
(a) culturing first cells which are first C-X-C chemokine receptor 4 (Cxcr4)-positive and KIT proto-oncogene receptor tyrosine kinase (KIT)-positive WD progenitor cell-like cells to provide second cells in a first medium containing (i) retinoic acid or a retinoic acid analog, (ii) a Wnt agonist, (iii) FGF9, and (iv) a ROCK inhibitor;
(b) culturing the second cells to provide third cells in a second medium comprising (i) retinoic acid or a retinoic acid analog, (ii) a Wnt agonist, (iii) a fibroblast cell growth factor, (iv) a ROCK inhibitor, and (v) a glial cell line-derived neurotrophic factor (GDNF) or a GDNF analog, and
(c) culturing the third cells obtained in a third medium comprising (i) retinoic acid or a retinoic acid analog, (ii) a Wnt agonist, (iii) a ROCK inhibitor, and (iv) GDNF or a GDNF analog.

30. A method according to claim 28, wherein the ureteric bud-like cells are obtained by:
(i) culturing first pluripotent stem cells as second pluripotent stem cells in a fourth medium comprising activin A or a tumor growth factor;
(ii) culturing the second pluripotent stem cells as third pluripotent stem cells in a second medium comprising a Wnt agonist, optionally further comprising a BMP signal pathway active substance;
(iii) culturing the third pluripotent stem cells as fourth cells in a third medium comprising (i) a retinoic acid or a retinoic acid analog, (ii) a fibroblast cell growth factor, and (iii) a TGFβ signal pathway inhibitor or a Wnt agonist,
(iv) culturing the fourth cells as fifth cells in a medium comprising (i) a retinoic acid or a retinoic acid analog, (ii) a Wnt agonist, and (iii) a fibroblast cell growth factor; and
(v) obtaining the C-X-C chemokine receptor 4 (Cxcr4)-positive and KIT proto-oncogene receptor tyrosine kinase (KIT)-positive cells from the fifth cells,
wherein the components in each of procedures (i)-(iv) can be the same substance or different substances.
